(19) United States
(12) Patent Application Publication
(10) Pub. No.: US 2023/0001029 A1
(43) Pub. Date: Jan. 5, 2023

(54) Combined Light Disinfection Device

(71) Applicant: Vyv, Inc., Latham, NY (US)

(72) Inventors: Robert Barron, Boulder, CO (US); Cori Winslow, Rensselaer, NY (US); Devanshi Jesrani, Troy, NY (US); Jonas Ciemny, Colonie, NY (US)

(21) Appl. No.: 17/856,512

(22) Filed: Jul. 1, 2022

Related U.S. Application Data
(60) Provisional application No. 63/217,472, filed on Jul. 1, 2021.

(51) Int. Cl. A61L 2/10; A61L 2/08

(52) U.S. Cl. CPC A61L 2/10; A61L 2/084; A61L 2202/11

(57) ABSTRACT

Apparatuses, systems, and methods for combined light disinfection are disclosed. In some examples, a light fixture may comprise a first light source configured to emit a first light comprising a wavelength in a range of 380-420 nanometers (nm) and a second light source configured to emit a second light comprising a wavelength in a range of 200-230 nm, wherein the first light source and the second light source are separated to prevent degradation, by the second light, of the first light source, and wherein the first light and the second light combine to form a disinfecting light configured to initiate inactivation of microorganisms.

### CROSS-REFERENCE TO RELATED APPLICATIONS

This application claims the benefit of U.S. Provisional Application No. 63/217,472, filed on Jul. 1, 2021. The above-referenced application is hereby incorporated by reference in its entirety.

### TECHNICAL FIELD

Aspects of the present disclosure generally relate to processes, systems, and apparatus for a combined light disinfection device.

### BACKGROUND OF THE INVENTION

Light-emitting devices are a primary requirement in most indoor occupied environments to provide illumination of the area, of tasks being completed in the area, and of the area's occupants and objects. Alternative light sources have been created with additional performance factors in mind that utilize emitted light or energy in different manners. Lighting fixtures and devices for horticulture, health, warmth, and disinfection have been demonstrated. In addition to being tuned for luminous efficacy of radiation, these lighting fixtures and devices are tuned to provide increased outputs of certain regions of radiation to accomplish the additional performance factor. In these lighting fixtures and devices that emit light for multiple functions, the light emissions can be balanced to achieve an acceptable level of each function. One of the functions can be general illumination (e.g., when the multiple-function lighting fixtures and devices are used in spaces occupied by humans), in which case, achieving a relatively high luminous efficacy of the emitted light is balanced not only against achieving desirable color characteristics of the emitted light, but also of achieving the one or more other functions to an acceptable or desired level.

Microorganism inactivation is a crucial practice required in many areas of both personal and environmental hygiene for the benefit of human health. Many methods are employed for a variety of situations where human or animal health factors may be improved by inactivation of bacteria, viruses, and microorganisms. Sickness and infection are the primary concerns of microorganism contamination through the many modes of intake of organisms into the human body from the environment. The human body may become sickened or infected by many different modes. Some modes may be due to internal imbalances of natural human microorganisms, but many problematic cases are caused by the transmission of microorganisms by either human to human contact or proximity, or by intake of microorganisms from the immediate environment.

The need to address the significant impact of viruses on the human population has become more apparent over the years. Because viruses are transferred between their hosts by transfer from surfaces or movement through the air, inactivation of viruses on surfaces and within air may be a contributing factor to decreasing the transmission of viruses. Wavelengths of light that can damage biomolecular structures of viruses are useful to damage the viruses and render them inactive and unable to infect host cells and replicate. One well known manifestation of this phenomenon is the use of ultraviolet light to inactivate viruses by damage to the nucleic acid and/or membrane of a virus rendering it unable to infect or replicate in host cells.

### BRIEF DESCRIPTION OF THE INVENTION

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary merely presents some concepts of the disclosure in a simplified form as a prelude to the description below.

An embodiment disclosed herein of an apparatus may comprise a first light source configured to emit a first light comprising a wavelength in a range of 380-420 nanometers (nm) and a second light source configured to emit a second light comprising a wavelength in a range of 200-230 nm, wherein the first light source and the second light source are separated to prevent degradation, by the second light, of the first light source, and wherein the first light and the second light combine to form a disinfecting light configured to initiate inactivation of microorganisms.

A light fixture disclosed herein may include a first light source configured to emit a first light comprising a wavelength in a range of 380-420 nanometers (nm), and a second light source configured to emit a second light comprising a wavelength in a range of 200-230 nm. In some examples, the first light source and the second light source may be separated to prevent degradation, by the second light, of the first light source, and the first light and the second light may combine to form a disinfecting light configured to initiate inactivation of microorganisms. In another example, the disinfecting light may be configured to initiate inactivation of microorganisms on a surface, in air, and/or in a liquid. In some examples, the first light source may include at least one LED and the second light source may include at least one LED or lamp. Alternatively, the second light source may not be an LED. In some examples, the light fixture may further include a lens or aperture coupled to the second light source configured to create a cutoff angle of the second light, and the cutoff angle may prevent inadvertent or dangerous eye exposure to the second light. In still another example, the light fixture may include a third light source configured to emit a third light comprising a wavelength from about 250 nm to 290 nm. In some examples, the first light and the second light may combine to form a white light, and the second light source may emit the second light for a dose of about 250 J/m2. In yet another example, the exposure time, or dosage, may minimize ozone generation. In one example, the ozone generation may be minimized to less than 0.05 parts per million. In some examples, the irradiance and ozone generation related to the first light and/or second light may be modulated to limit exposure time or dosage to far-UVC emissions.

A system disclosed herein may include a first light source configured to emit a first light comprising a wavelength in a range of 380-420 nm and a second light source configured to emit a second light comprising a wavelength in a range of 200-230 nm in which the first light and the second light may combine to form a disinfecting light configured to initiate the inactivation of microorganisms, and may include an integrated control system configured to determine a minimum dosage of the disinfecting light, and configured to determine a maximum dosage of UV light exposure. In some examples, the system may also include a third light source configured to emit a third light comprising a wavelength of about 254 nm, or a white light. In other examples, the disinfecting light may be configured to initiate the inactivation of microorganisms on a surface, in air, and/or in a liquid. In yet other examples, the integrated control system control may be configured to independently control the first light source, and the second light source, and may be further configured to limit the first light emission and the second light emission based upon an emitted wavelength and intensity of the first light source and the second light source. In one example, the system may include a filter coupled to the second light source and configured to block wavelengths outside of the range 200-230 nm. In some examples, the second light source may be configured to pulse the emitted light. In another example, the first light source may include a plurality of LEDs and include at least one LED or lamp. Alternatively, the second light source may not be an LED.

A method for inactivating microorganisms disclosed herein may include the steps of providing a first light source configured to emit a first light comprising a wavelength in a range of 380-420 nm, providing a second light source configured to emit a second light comprising a wavelength in a range of 200-230 nm, separating the first light source from the second light source to prevent degradation by the second light of the first light source, combining the first light and the second light to form a disinfecting light configured to inactivate microorganisms on a surface, in air, and/or in a liquid, initiating inactivation of microorganisms. In another example, the method may include a second light source that emits the second light for an exposure time of about 8-hours or less, and the exposure time may minimize ozone generation, and the ozone generation may be less than 0.05 parts per million (ppm). In other examples, the method may further include the step of providing a third light source configured to emit a third light comprising a wavelength of about 254 nm. In some examples, the irradiance and ozone generation related to the first light and/or second light may be modulated to limit exposure time or dosage to far-UVC emissions.

These and additional features will be appreciated with the benefit of the disclosures discussed in further detail below.

### BRIEF DESCRIPTION OF THE DRAWINGS

FIGS. 1-43 illustrate exemplary configurations of combined light disinfection devices in accordance with the present disclosure.

FIGS. 44-47 illustrate example flowcharts for various processes/algorithms that may be performed in accordance with the present disclosure.

FIG. 48 illustrates an example computing device that may be used to implement one or more processes, algorithms, or devices in accordance with the present disclosure.

FIG. 49 illustrates an American National Standards Institute (ANSI) C78.377-2017 White Light Standards diagram using the CIE 1931 xy coordinate system with accepted x-y coordinates at selected CCTs that are color coordinate ranges for light-emitting devices in accordance with the present disclosure.

FIGS. 50-51 illustrate International Commission on Illumination (CIE) 1931 xy color space diagrams.

### DETAILED DESCRIPTION

In the following description of the various examples, reference is made to the accompanying drawings, which form a part hereof, and in which is shown by way of illustration, various examples of the disclosure that may be practiced. It is to be understood that other examples may be utilized.

Wavelengths of visible light in the violet range, 380-420 nanometer (nm) (e.g., 405 nm), may have a lethal effect on microorganisms. As used herein, the term “microorganisms” encompasses at least viruses (including enveloped and non-enveloped viruses), bacteria (including gram positive and gram-negative bacteria), bacterial endospores, yeasts, molds, and filamentous fungi. For example, Escherichia coli (E. coli), Salmonella, Methicillin-resistant Staphylococcus aureus (MRSA), and Clostridium difficile may be susceptible to 380-420 nm light. Such wavelengths may initiate a photoreaction within non-iron porphyrin molecules found in some microorganisms. The non-iron porphyrin molecules may be photoactivated and may react with other cellular components to produce Reactive Oxygen Species (ROS). ROS may cause irreparable cell damage and eventually destroy, kill, or otherwise inactivate cells of some microorganisms. Non-iron porphyrins are specific to microorganisms only therefore, because humans, plants, and/or animals do not contain these same non-iron porphyrin molecules, this technique may be completely safe for human, plant, and animal exposure. Light in the 380-420 nm wavelength may be effective against every type of bacteria, although it may take different amounts of time or dosages depending upon the species. 380-420 nm light (e.g., 405 nm), may be effective against all gram-negative and gram-positive bacteria to some extent over a period of time. It can also be effective against many varieties of fungi.

In some examples, visible light in the violet range, 380-420 nanometer (nm) (e.g., 405 nm), may decrease viral load on a surface, in the air, or in a liquid. Viruses may rely on surface bacteria, yeast, mold, or fungi as hosts. By decreasing surface bacteria, yeast, mold, or fungi count, for example, by using 380-420 nm light, the viral load may also be decreased. In some examples, viruses may be susceptible to reactive oxygen species. Viral load may decrease when the viruses are surrounded by a medium that can produce reactive oxygen species to inactivate viruses. In some examples, the medium may comprise fluids or droplets that comprise bacteria or other particles that produce oxygen reactive species. In some examples, the medium may comprise respiratory droplets, saliva, feces, organic rich media, and/or blood plasma.

Proteins and genomic material may be major targets for UV inactivation in microorganisms. Photons from UV may be directly absorbed by pathogenic DNA leading to the formation of dimers between pyrimidine residues. These dimers may lead to the production of two DNA photoproducts, namely cyclobutane-pyrimidine dimers (CPD's) and 6-4-photoproducts (6-4 PP's), that cause cytotoxic and lethal mutagenic effects, respectively. The photoproducts may distort the DNA helix and block DNA replication, transcription and translation. This may result in mutations and biochemical alterations causing bacterial cell death and viral inactivation. A second pathogen kill mechanism may involve oxidation of nucleic acids, proteins and lipids triggered by the production of reactive oxygen species (ROS) like single oxygen and hydrogen radicals when the exogenous or endogenous photosensitizers are irradiated by UV. Accumulation of DNA damage may lead to loss of metabolic cell activity and the inability to generate energy. The overall structure of nucleic acids and proteins may be altered, thus interfering with vital biological functions. Aggregated membrane proteins may undergo heavy stress, resulting in impairment of protein translation. Generation of ROS also may lead to fatty acid oxidation at the lipid bilayer causing damage to cell membranes, cell walls and proteins.

Inactivation mechanisms may involve absorption of UV by nitrogenous bases in DNA that may cause damage to nucleic acids in fungal cells. Formation of reactive oxygen species may lead to the oxidation of DNA bases causing physiological dysfunction. In addition, oxidation of the cellular components may cause accelerated penetration and absorption of UV light into the fungal cells which may promote cell disintegration. Oxidation of the cell membrane and cell wall may cause cell damage, membrane instability and alteration in membrane permeability affecting cell metabolism and osmotic pressure balance. Decrease in membrane potential may disrupt essential membrane transport processes. UV radiation may also damage proteins that play a significant role in stabilizing the cell membrane. Morphological damage to fungal cells may lead to release of the intracellular components that may be vital for fungal cell function and communication.

In some examples, inactivation, in relation to microorganism death, may include control and/or reduction in microorganism colonies or individual cells when exposed to disinfecting light for a certain duration. Light may be utilized for inactivation using a peak wavelength of light, or in some examples, multiple peak wavelengths, in a range of approximately 380 nm to 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. Such light may damage viral capsids, nucleic acids, and also lead to the degradation of the genomes. Destruction of nucleic acids and genomes may prevent replication function in host cells leading to loss of infectivity. Unsaturated lipids and major envelope proteins may form cross links that lead to loss of proteins causing conformational changes in the capsid structure. This may affect the stability of a virus in host cell receptors. Protein mediated binding, injection or replication functions may be impaired. Significant changes in molecular mass and charge of proteins may occur, which may hinder viral entry and cytopathic effects.

The electromagnetic spectrum may be harnessed within devices, systems, and apparatuses to utilize its functions for benefit of humans/animals. Most portions of the electromagnetic spectrum are not visible with the exception of the visible light spectrum within the range of approximately 380 nm to 750 nm. The ultraviolet spectrum comprises the energy within the range of approximately 100 nm to 400 nm and is generally not visible. Due to the lack of (or minimal) visibility, ultraviolet light, may be integrated into general lighting with minimal adjustments to the existing lighting for general illumination.

General illumination may include lighting produced to illuminate at least a portion of an indoor area for areas occupied by users or uses illumination for the completion of tasks. General illumination devices may include overhead ceiling fixtures, table lamps, floor lamps, task lighting, etc. General illumination may be white light with certain defining characteristics. General illumination may be created from wavelengths in the visible light spectrum mixed together to form a white light.

There may be a minimum irradiance required to hit the surface to cause microbial inactivation. A minimum irradiance of light (e.g., in the 380-420 nm wavelength) on a surface may cause microbial inactivation. For example, a minimum irradiance of 0.02 milliwatts per square centimeter (mW/cm²) may cause microbial inactivation on a surface over time. In some examples, an irradiance of 0.05 mW/cm² may inactivate microorganisms on a surface, but higher values such as 0.1 mW/cm², 0.5 mW/cm², 1 mW/cm², or 2 mW/cm² may be used for quicker microorganism inactivation. In some examples, even higher irradiances may be used over shorter periods of time, e.g., 3 to 10 mW/cm². In other examples, a minimum irradiance may be, for example, at least, greater than, less than, equal to, or any number in between about 0.01 mW/cm², 0.02 mW/cm², 0.03 mW/cm², 0.04 mW/cm², 0.05 mW/cm², 0.06 mW/cm², 0.07 mW/cm², 0.08 mW/cm², 0.09 mW/cm², 0.1 mW/cm², 0.1 mW/cm², 0.2 mW/cm², 0.3 mW/cm², 0.4 mW/cm², 0.5 mW/cm², 0.6 mW/cm², 0.7 mW/cm², 0.8 mW/cm², 0.9 mW/cm², 1.0 mW/cm², 1.1 mW/cm², 1.2 mW/cm², 1.3 mW/cm², 1.4 mW/cm², 1.5 mW/cm², 1.6 mW/cm², 1.7 mW/cm², 1.8 mW/cm², 1.9 mW/cm², 2.0 mW/cm², 2.1 mW/cm², 2.2 mW/cm², 2.3 mW/cm², 2.4 mW/cm², 2.5 mW/cm², 2.6 mW/cm², 2.7 mW/cm², 2.8 mW/cm², 2.9 mW/cm², 3.0 mW/cm², 3.1 mW/cm², 3.2 mW/cm², 3.3 mW/cm², 3.4 mW/cm², 3.5 mW/cm², 3.6 mW/cm², 3.7 mW/cm², 3.8 mW/cm², 3.9 mW/cm², 4.0 mW/cm², 4.1 mW/cm², 4.2 mW/cm², 4.3 mW/cm², 4.4 mW/cm², 4.5 mW/cm², 4.6 mW/cm², 4.7 mW/cm², 4.8 mW/cm², 4.9 mW/cm², 5.0 mW/cm², 5.1 mW/cm², 5.2 mW/cm², 5.3 mW/cm², 5.4 mW/cm², 5.5 mW/cm², 5.6 mW/cm², 5.7 mW/cm², 5.8 mW/cm², 5.9 mW/cm², 6.0 mW/cm², 6.1 mW/cm², 6.2 mW/cm², 6.3 mW/cm², 6.4 mW/cm², 6.5 mW/cm², 6.6 mW/cm², 6.7 mW/cm², 6.8 mW/cm², 6.9 mW/cm², 7.0 mW/cm², 7.1 mW/cm², 7.2 mW/cm², 7.3 mW/cm², 7.4 mW/cm², 7.5 mW/cm², 7.6 mW/cm², 7.7 mW/cm², 7.8 mW/cm², 7.9 mW/cm², 8.0 mW/cm², 8.1 mW/cm², 8.2 mW/cm², 8.3 mW/cm², 8.4 mW/cm², 8.5 mW/cm², 8.6 mW/cm², 8.7 mW/cm², 8.8 mW/cm², 8.9 mW/cm², 9.0 mW/cm², 9.1 mW/cm², 9.2 mW/cm², 9.3 mW/cm², 9.4 mW/cm², 9.5 mW/cm², 9.6 mW/cm², 9.7 mW/cm², 9.8 mW/cm², 9.9 mW/cm², and 10.0 mW/cm². Example light emitters disclosed herein may be configured to produce light with such irradiances at any given surface, in air, or in a liquid.

In some examples, light for microbial inactivation may include radiometric energy sufficient to inactive at least one microorganism population, or in some examples, a plurality of microorganism populations. One or more disinfecting lighting element(s) may have some minimum amount of radiometric energy (e.g., 20 mW) measured from 380-420 nm light. In one example, one or more lighting element(s) may emit some minimum amount of radiometric energy measured from, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm. In another example, the one or more lighting element(s) may emit a minimum amount of radiometric energy measured from, for example, at least, greater than, less than, equal to, or any number in between about 10 mW, 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 105 mW, 110 mW, 115 mW, 120 mW, 125 mW, 130 mW, 135 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, 360 mW, 365 mW, 370 mW, 375 mW, 380 mW, 385 mW, 390 mW, 395 mW, 400 mW, 405 mW, 410 mW, 415 mW, 420 mW, 425 mW, 430 mW, 435 mW, 440 mW, 445 mW, 450 mW, 455 mW, 460 mW, 465 mW, 470 mW, 475 mW, 480 mW, 485 mW, 490 mW, 495 mW, 500 mW, 505 mW, 510 mW, 515 mW, 520 mW, 525 mW, 530 mW, 535 mW, 540 mW, 545 mW, 550 mW, 555 mW, 560 mW, 565 mW, 570 mW, 575 mW, 580 mW, 585 mW, 590 mW, 595 mW, 600 mW, 605 mW, 610 mW, 615 mW, 620 mW, 625 mW, 630 mW, 635 mW, 640 mW, 645 mW, 650 mW, 655 mW, 660 mW, 665 mW, 670 mW, 675 mW, 680 mW, 685 mW, 690 mW, 695 mW, 700 mW, 705 mW, 710 mW, 715 mW, 720 mW, 725 mW, 730 mW, 735 mW, 740 mW, 745 mW, 750 mW, 755 mW, 760 mW, 765 mW, 770 mW, 775 mW, 780 mW, 785 mW, 790 mW, 795 mW, 800 mW, 805 mW, 810 mW, 815 mW, 820 mW, 825 mW, 830 mW, 835 mW, 840 mW, 845 mW, 850 mW, 855 mW, 860 mW, 865 mW, 870 mW, 875 mW, 880 mW, 885 mW, 890 mW, 895 mW, 900 mW, 905 mW, 910 mW, 915 mW, 920 mW, 925 mW, 930 mW, 935 mW, 940 mW, 945 mW, 950 mW, 955 mW, 960 mW, 965 mW, 970 mW, 975 mW, 980 mW, 985 mW, 990 mW, 995 mW, 1000 mW, 1005 mW, 1010 mW, 1015 mW, 1020 mW, 1025 mW, 1030 mW, 1035 mW, 1040 mW, 1045 mW, 1050 mW, 1055 mW, 1060 mW, 1065 mW, 1070 mW, 1075 mW, 1080 mW, 1085 mW, 1090 mW, 1095 mW, 1100 mW, 1105 mW, 1110 mW, 1115 mW, 1120 mW, 1125 mW, 1130 mW, 1135 mW, 1140 mW, 1145 mW, 1150 mW, 1155 mW, 1160 mW, 1165 mW, 1170 mW, 1175 mW, 1180 mW, 1185 mW, 1190 mW, 1195 mW, 1200 mW, 1205 mW, 1210 mW, 1215 mW, 1220 mW, 1225 mW, 1230 mW, 1235 mW, 1240 mW, 1245 mW, 1250 mW, 1255 mW, 1260 mW, 1265 mW, 1270 mW, 1275 mW, 1280 mW, 1285 mW, 1290 mW, 1295 mW, 1300 mW, 1305 mW, 1310 mW, 1315 mW, 1320 mW, 1325 mW, 1330 mW, 1335 mW, 1340 mW, 1345 mW, 1350 mW, 1355 mW, 1360 mW, 1365 mW, 1370 mW, 1375 mW, 1380 mW, 1385 mW, 1390 mW, 1395 mW, 1400 mW, 1405 mW, 1410 mW, 1415 mW, 1420 mW, 1425 mW, 1430 mW, 1435 mW, 1440 mW, 1445 mW, 1450 mW, 1455 mW, 1460 mW, 1465 mW, 1470 mW, 1475 mW, 1480 mW, 1485 mW, 1490 mW, 1495 mW, 1500 mW, 1505 mW, 1510 mW, 1515 mW, 1520 mW, 1525 mW, 1530 mW, 1535 mW, 1540 mW, 1545 mW, 1550 mW, 1555 mW, 1560 mW, 1565 mW, 1570 mW, 1575 mW, 1580 mW, 1585 mW, 1590 mW, 1595 mW, 1600 mW, 1605 mW, 1610 mW, 1615 mW, 1620 mW, 1625 mW, 1630 mW, 1635 mW, 1640 mW, 1645 mW, 1650 mW, 1655 mW, 1660 mW, 1665 mW, 1670 mW, 1675 mW, 1680 mW, 1685 mW, 1690 mW, 1695 mW, 1700 mW, 1705 mW, 1710 mW, 1715 mW, 1720 mW, 1725 mW, 1730 mW, 1735 mW, 1740 mW, 1745 mW, 1750 mW, 1755 mW, 1760 mW, 1765 mW, 1770 mW, 1775 mW, 1780 mW, 1785 mW, 1790 mW, 1795 mW, 1800 mW, 1805 mW, 1810 mW, 1815 mW, 1820 mW, 1825 mW, 1830 mW, 1835 mW, 1840 mW, 1845 mW, 1850 mW, 1855 mW, 1860 mW, 1865 mW, 1870 mW, 1875 mW, 1880 mW, 1885 mW, 1890 mW, 1895 mW, 1900 mW, 1905 mW, 1910 mW, 1915 mW, 1920 mW, 1925 mW, 1930 mW, 1935 mW, 1940 mW, 1945 mW, 1950 mW, 1955 mW, 1960 mW, 1965 mW, 1970 mW, 1975 mW, 1980 mW, 1985 mW, 1990 mW, 1995 mW, and 2000 mW.

Dosage (measured in Joules/cm²) may be another metric for determining an appropriate irradiance for microbial inactivation over a period of time. Table 1 below shows example correlations between irradiance in mW/cm² and Joules/cm² based on different exposure times. These values are examples and many others may be possible.

TABLE 1

| Irradiance | Exposure | Dosage |
|---|---|---|
| (mW/cm²) | Time (hours) | (Joules/cm²) |
| 0.02 | 1 | 0.072 |
| 0.02 | 24 | 1.728 |
| 0.02 | 250 | 18 |
| 0.02 | 500 | 36 |
| 0.02 | 1000 | 72 |
| 0.05 | 1 | 0.18 |
| 0.05 | 24 | 4.32 |
| 0.05 | 250 | 45 |
| 0.05 | 500 | 90 |
| 0.05 | 1000 | 180 |
| 0.1 | 1 | 0.36 |
| 0.1 | 24 | 8.64 |
| 0.1 | 250 | 90 |
| 0.1 | 500 | 180 |
| 0.1 | 1000 | 360 |
| 0.5 | 1 | 1.8 |
| 0.5 | 24 | 43.2 |
| 0.5 | 250 | 450 |
| 0.5 | 500 | 900 |
| 0.5 | 1000 | 1800 |
| 1 | 1 | 3.6 |
| 1 | 24 | 86.4 |
| 1 | 250 | 900 |
| 1 | 500 | 1800 |
| 1 | 1000 | 3600 |

Microbial inactivation may comprise a target reduction in microorganism population(s) (e.g., 1-Log 10 reduction, 2-Log 10 reduction, 99% reduction, or the like). Table 2 shows example dosages recommended for the inactivation (measured as 1-Log 10 reduction in population) of different microorganism species using narrow spectrum 405 nm light. Example dosages and other calculations shown herein may be determined based on laboratory settings. Real world applications may require dosages that may differ from example laboratory data. Other dosages of 380-420 nm (e.g., 405 nm) light may be used with other bacteria not listed below.

TABLE 2

| | Recommended Dose |
|---|---|
| | (J/cm²) for 1-Log |
| | Reduction in |
| Microorganism | Microorganism |
| Staphylococcus aureus | 20 |
| MRSA | 20 |
| Pseudomonas aeruginosa | 45 |
| Escherichia coli | 80 |
| Enterococcus faecalis | 90 |

Equation 1 may be used in order to determine irradiance, dosage, or time using one or more data points from Table 1 and Table 2:

$$\frac{Irradiance ( \frac{mW}{{cm}^{2}} )}{1000} * Time ( s ) = Dosage ( \frac{J}{{cm}^{2}} ) \qquad Equation 1$$

Irradiance may be determined based on dosage and time. For example, if a dosage of 30 Joules/cm² is recommended and the object desired to be disinfected is exposed to light overnight for 8 hours, the irradiance may be approximately 1 mW/cm². If a dosage of 50 Joules/cm² is recommended and the object desired to be disinfected is exposed to light for 48 hours, a smaller irradiance of only approximately 0.3 mW/cm² may be sufficient.

Time may be determined based on irradiance and dosage. For example, a device may be configured to emit an irradiance of disinfecting energy (e.g., 0.05 mW/cm²) and a target bacteria may require a dosage of 20 Joules/cm² to kill the target bacteria. Disinfecting light at 0.05 mW/cm² may have a minimum exposure time of approximately 4.6 days to achieve the dosage of 20 Joules/cm². Dosage values may be determined by a target reduction in microorganisms. Once the microorganism count is reduced to a desired amount, disinfecting light may be continuously applied to keep the microorganism counts down.

Radiant flux (e.g., radiant power, radiant energy), measured in Watts, is the total power from a light source. Irradiance is the power per unit area at a distance away from the light source. In some examples, the target irradiance on a target surface from the light source may be 10 mW/cm². A 10 mW/cm² target irradiance may be provided, for example, by a light source with a radiant flux of 10 mW located 1 cm from the target surface. In another example, a light source may be located 5 cm from the target surface. With a target irradiance of 10 mW/cm², the light source may be configured to produce a radiant flux approximately 250 mW. These calculations may be approximately based on the inverse square law, as shown in Equation 1, where the excitation light source may be assumed to be a point source, E is the irradiance, I is the radiant flux, and r is the distance from the excitation light source to a target surface.

$$E ≅ \frac{I}{{r}^{2}} \qquad Equation 2$$

In some examples, different wavelengths of light may have different effects on different microorganisms. The tables below illustrate example data related to application of various wavelengths of light on various microorganisms. For example, tables 3-7 summarize the recommended dose response for the inactivation of microorganisms at different log levels when exposed to wavelengths of 405 nm, 222 nm and 254 nm light. Inactivation may comprise a target reduction in microorganism population(s) (e.g., 1-Log 10 reduction, 2-Log 10 reduction, 99% reduction, or the like).

Table 3 shows example dosages measured in J/cm² which may be used for the inactivation (at different log levels) of different microorganisms using 222 nm light.

TABLE 3

| | Recommended Dose (J/cm²) for Reduction | | | |
|---|---|---|---|---|
| | in Microorganisms at 222 nm | | | |
| Microorganism | 1-log | 2-log | 3-log | 4-log |
| Staphylococcus | 9.3 × 10⁻³ | 1.15 × 10⁻² | 1.38 × 10⁻² | 1.78 × 10⁻² |
| aureus | | | | |
| Pseudomonas | 3.1 × 10⁻³ | 4.8 × 10⁻³ | 5.9 × 10⁻³ | 7.5 × 10⁻³ |
| aeruginosa | | | | |
| Aspergillus | 9 × 10⁻² | 0.220 | 0.325 | 0.430 |
| niger | | | | |

Table 4 shows example dosages measured in J/cm² which may be used for the inactivation (at different log levels) of different microorganisms using 254 nm light.

TABLE 4

| | Recommended Dose (J/cm²) for Reduction | | | |
|---|---|---|---|---|
| | in Microorganisms at 254 nm | | | |
| Microorganism | 1-log | 2-log | 3-log | 4-log |
| Staphylococcus | 4.4 × 10⁻³ | 6.0 × 10⁻³ | 7.3 × 10⁻³ | 9.5 × 10⁻³ |
| aureus | | | | |
| Streptococcus | 6.6 × 10⁻³ | 8.8 × 10⁻³ | 9.9 × 10⁻³ | 1.12 × 10⁻² |
| faecalis | | | | |
| Pseudomonas | 8 × 10⁻⁴ | 1.6 × 10⁻³ | 2.3 × 10⁻³ | 3.1 × 10⁻³ |
| aeruginosa | | | | |
| Escherichia coli | 3 × 10⁻³ | 4.8 × 10⁻³ | 6.7 × 10⁻³ | 8.4 × 10⁻³ |
| Aspergillus | 0.115 | 0.245 | 0.370 | 0.560 |
| niger | | | | |

Table 5 shows example dosages measured in J/cm² which may be used for the inactivation (at different log levels) of different microorganisms using 222 nm light.

TABLE 5

| | Recommended Dose (J/cm²) for Reduction | | | |
|---|---|---|---|---|
| | in Microorganisms at 222 nm | | | |
| Microorganism | Type | Reduction | Light dosage | Medium |
| Influenza A | Enveloped | 1 log | 1.3 × 10⁻³ | Airborne |
| | | 2 log | 2.6 × 10⁻³ | |
| | | 3 log | 3.8 × 10⁻³ | |
| HCoV 229-E | Enveloped | 1 log | 5.6 × 10⁻⁴ | Airborne |
| | | 2 log | 1.1 × 10⁻³ | |
| | | 3 log | 1.7 × 10⁻³ | |
| HCoV OCV3 | Enveloped | 1 log | 3.9 × 10⁻⁴ | Airborne |
| | | 2 log | 7.8 × 10⁻⁴ | |
| | | 3 log | 1.2 × 10⁻³ | |

Table 6 shows example dosages measured in J/cm² which may be used for the inactivation (at different log levels) of different microorganisms using 254 nm light.

TABLE 6

| | Recommended Dose (J/cm²) for Reduction in | | | |
|---|---|---|---|---|
| | Microorganisms at 254 nm | | | |
| Microorganism | Type | Reduction | Light dosage | Medium |
| Influenza A | Enveloped | 1 log | 1.04 × 10⁻³ | Airborne |
| | | 1.4 log | 1.48 × 10⁻³ | |
| Influenza A | Enveloped | 4.08 log to | 1.8 | Solid |
| | | 5.75 log | | |
| SARS CoV | Enveloped | 3.4 log to | 0.15 | Liquid |
| | | 3.6 log | 1.4 | |
| | | 4 log | | |
| SARS CoV | Enveloped | 4 log | 0.12 | Solid |
| SARS CoV2 | Enveloped | 5.7 log | 1.6 × 10⁻² | Liquid |
| MS₂ | Non-enveloped | 1 log | 3.4-4.2 × 10⁻⁴ | Airborne |
| bacteriophage | | 2 log | 8-9.1 × 10⁻⁴ | |
| MS₂ | Non-enveloped | 1 log | 1.86- | Liquid |
| bacteriophage | | 4 log | 2.57 × 10⁻² | |
| | | | 0.12 | |
| MS2 | Non-enveloped | 1 log | 3.2 × 10⁻³ | Solid |
| bacteriophage | | 3 log to | 4.32-7.2 | |
| | | 4 log | | |
| FCV | Non-enveloped | 1 log | 5-6 × 10⁻³ | Liquid |
| | | 4 log | 0.04 | |
| FCV | Non-Enveloped | 2.12 log- | 0.2 | Solid |
| | | 4.46 log | | |
| Adenovirus type | Non-enveloped | 1 log | 5.5 × 10⁻² | Liquid |
| 40 | | 2 log | 0.105 | |
| | | 3 log | 0.155 | |
| Rotavirus | Non-enveloped | 1 log | 2.0 × 10⁻² | Liquid |
| | | 2 log | 8.0 × 10⁻² | |
| | | 3 log | 0.140 | |
| | | 4 log | 0.2 | |
| Polio virus 1 | Non-enveloped | 1 log | 7 × 10⁻³ | Liquid |
| | | 2 log | 1.7 × 10⁻² | |
| | | 3 log | 2.8 × 10⁻² | |
| | | 4 log | 3.7 × 10⁻² | |
| Hepatitis A | Non-enveloped | 1 log | 5.5 × 10⁻³ | Liquid |
| | | 2 log | 9.8 × 10⁻³ | |
| | | 3 log | 1.5 × 10⁻² | |
| | | 4 log | 2.1 × 10⁻² | |
| Murine | Non-enveloped | 1 log | 1 × 10⁻² | Liquid |
| norovirus | | | | |

Table 7 shows example dosages measured in J/cm² which may be used for the inactivation (at different log levels) of different microorganisms using 405 nm light.

TABLE 7

| | Recommended Dose (J/cm²) for Reduction | | | |
|---|---|---|---|---|
| | in Microorganisms at 405 nm | | | |
| Microorganism | Type | Reduction | Light dosage | Medium |
| SARS CoV2 | Enveloped | 1 log | 3.9 × 10⁻⁴ | Airborne |
| phi6 | Enveloped | 1 log | 430 | Liquid |
| | | 3 log | 1300 | |
| Bacteriophage | Non-Enveloped | 3 log | 300 | Liquid |
| sigma C31 | | 5 log | 500 | |
| | | 7 log | 1400 | |
| FCV | Non-enveloped | 3.9 log | 2800 | Liquid |

Because one or more of the wavelengths of light described above fall within the ultraviolet range, and because ultraviolet light may have adverse effects on humans and animals, methods to ensure safe exposure to such light are described herein. The American Conference of Governmental Industrial Hygienists (ACGIH) sets forth Ultraviolet Radiation exposure limits. ACGIH sets “Threshold Limit Values” (TLVs) for different frequencies of electromagnetic radiation, including ultraviolet radiation. Ultraviolet radiation may include light having wavelengths between 180 and 400 nanometers. In some examples, one or more lighting elements may emit light having wavelengths, for example, at least, greater than, less than, equal to, or any number in between about 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm and 400 nm. These exposure limits may represent the maximum level of safe exposure in an up to 8-hour period.

TABLE 8
UV relative spectral effectiveness per
wavelength weighting table

| Wavelength | TLV | Relative Spectral |
|---|---|---|
| (nm) | (J/m²) | Effectiveness, S(λ) |
| 180 | 2500 | 0.012 |
| 190 | 1600 | 0.019 |
| 200 | 1000 | 0.03 |
| 205 | 590 | 0.051 |
| 210 | 400 | 0.075 |
| 215 | 320 | 0.095 |
| 220 | 250 | 0.12 |
| 225 | 200 | 0.15 |
| 230 | 160 | 0.19 |
| 235 | 130 | 0.24 |
| 240 | 100 | 0.3 |
| 250 | 70 | 0.43 |
| 260 | 46 | 0.65 |
| 270 | 30 | 1 |
| 280 | 34 | 0.88 |
| 290 | 47 | 0.64 |
| 300 | 100 | 0.3 |
| 310 | 2000 | 0.015 |
| 320 | 2.9 × 104 | 0.001 |
| 330 | 7.3 × 104 | 0.00041 |
| 340 | 1.1 × 10⁵ | 0.00028 |
| 350 | 1.5 × 10⁵ | 0.0003 |
| 360 | 2.3 × 10⁵ | 0.00013 |
| 370 | 3.2 × 10⁵ | 0.000093 |
| 380 | 4.7 × 10⁵ | 0.000064 |
| 385 | 5.7 × 10⁵ | 0.000053 |
| 390 | 6.8 × 10⁵ | 0.000044 |
| 395 | 8.3 × 10⁵ | 0.000036 |
| 400 | 1.0 × 10⁶ | 0.00003 |

Using table 8, exposure limits for a far-UVC emission source may be determined. For example, an emission source emitting primarily at 220 nm may deliver a dose of up to 250 J/m² (25 mJ/cm²) in an 8-hour period. An emission source emitting primarily at 225 nm may deliver a dose of up to 200 J/m² (20 mJ/cm²). Interpolating these values may provide a dose of up to 220 J/m² (22 mJ/cm²) for an emission source at 222 nm.

Using table 8 and equation 3 below, an example maximum exposure duration for an emission source at a given irradiance may be determined. In equation 3 below, Tₘₐₓ may be an example maximum exposure time in seconds. E_{eff} may be the effective irradiance relative to a monochromatic source at 270 nm, whose value may be determined by referencing table 8. E_{eff} for a particular wavelength may be determined based on the irradiance of that particular wavelength multiplied by the Relative Spectral Effectiveness for that particular wavelength as set forth in table 8. For example, an emission source emitting primarily at 225 nm at an irradiance of 0.00066 mW/cm² may have an E_{eff} of about 0.0001 mW/cm² (the irradiance of 0.00066 mW/cm² multiplied by the Relative Spectral Effectiveness of 0.15 per table 8:0.00066 mW/cm²*0.15=0.0001 mW/cm²). Based on equation 3 below, the example emission source may have an allowable exposure duration of about 8-hours (0.003 J/cm²/(0.0001 mW/cm²*1 W/1000 mW)=3000 seconds*(1 hr/3600 seconds)=8.33 hrs). Table 9 illustrates example maximum exposure times for respective effective irradiances per equation 3 and table 8.

$${t}_{max} [ s ] = \frac{0.003 [ \frac{J}{{cm}^{2}} ]}{{E}_{eff} [ \frac{W}{{cm}^{2}} ]} \qquad Equation 3$$

TABLE 9
Exposure duration for given Actinic UV
Radiation Effective Irradiances

| Duration of | Effective Irradiance |
|---|---|
| Exposure Per Day | E_{eff} (mW/cm2) |
| 8 hours | 0.0001 |
| 4 hours | 0.0002 |
| 2 hours | 0.0004 |
| 1 hour | 0.0008 |
| 30 minutes | 0.0017 |
| 15 minutes | 0.0033 |
| 10 minutes | 0.005 |
| 5 minutes | 0.01 |
| 1 minute | 0.05 |
| 1 second | 3 |
| 0.1 second | 30 |

The ACGIH has proposed modifying the Relative Spectral Effectiveness of far-UVC wavelengths to provide longer exposure times at the same irradiance, and higher irradiance for the same exposure time. Table 10 illustrates example modified exposure limits.

TABLE 10
proposed ACGIH exposure limits

| Wavelength | TLV | Relative Spectral |
|---|---|---|
| (nm) | (J/m²) | Effectiveness, S(λ) |
| 180 | 16300 | 0.00184 |
| 190 | 16300 | 0.00184 |
| 200 | 16300 | 0.00184 |
| 205 | 16300 | 0.00184 |
| 210 | 10233 | 0.00293 |
| 215 | 4732 | 0.00634 |
| 220 | 2188 | 0.0137 |
| 225 | 1012 | 0.0297 |
| 230 | 468 | 0.0625 |
| 235 | 216 | 0.136 |
| 240 | 100 | 0.3 |
| 250 | 70 | 0.43 |
| 260 | 46 | 0.65 |
| 270 | 30 | 1 |
| 280 | 34 | 0.88 |
| 290 | 47 | 0.64 |
| 300 | 100 | 0.3 |
| 310 | 2000 | 0.015 |
| 320 | 2.9 × 104 | 0.001 |
| 330 | 7.3 × 104 | 0.00041 |
| 340 | 1.1 × 10⁵ | 0.00028 |
| 350 | 1.5 × 10⁵ | 0.0003 |
| 360 | 2.3 × 10⁵ | 0.00013 |
| 370 | 3.2 × 10⁵ | 0.000093 |
| 380 | 4.7 × 10⁵ | 0.000064 |
| 385 | 5.7 × 10⁵ | 0.000053 |
| 390 | 6.8 × 10⁵ | 0.000044 |
| 395 | 8.3 × 10⁵ | 0.000036 |
| 400 | 1.0 × 10⁶ | 0.00003 |

UV radiation may be categorized into groups based on wavelength range i.e., UVA (320 nm to 400 nm), UVB (280 nm-320 nm), UVC (200 nm and 280 nm) and UVV (100 nm-200 nm). 222 nm light may penetrate up to 3 microns into cells. Since the diameter of pathogenic organisms may be between 0.1 to 1 micron, 222 nm light may effectively penetrate and destroy these microbes. However, human cells have an average diameter of greater than 40 microns. Thus, 222 nm light may not penetrate deep into human cells beneath the outer layer of skin. The 222 nm UV light photons may be absorbed by the cytoplasmic proteins and other biomolecules before they can reach the nuclei, which may prevent DNA damage. 222 nm light may also be absorbed by the tear layer of the eye, which may block photon penetration into corneal tissue. Thus, 222 nm may not cause any molecular damage to human/animal skin and eye. Unlike 222 nm light, 254 nm light may cause high cytotoxic and mutagenic DNA lesions and may produce a 1000-fold higher killing effect on human/animal fibroblasts. Photoproduct induction and photoinduced genotoxicity in epidermal DNA may occur. Inflammatory trigger responses, skin tumors and eye damage may be among possible consequences of irradiation with 254 nm UV light.

In some examples, it may be possible to effectively illuminate a full-size room with 222 nm light at an irradiance at or below the threshold limit values for skin for a continuous 8-hour exposure as directed by the American Conference of Governmental Industrial Hygienists. This level of irradiance may provide reductions in pathogen load of 90% or greater. In some examples, UV may be effective against common airborne viruses such as SARS-CoV-2. In other examples, the 222 nm light may be required for an exposure time of, for example, at least, greater than, less than, equal to, or any number in between about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 and 72 hours. In one example, the light used may have a wavelength of, for example, at least, greater than, less than, equal to, or any number in between about 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, and 235 nm.

In some examples, UVC wavelengths of light may cause ozone generation. In some examples, high-voltage power supplies and/or electrical contacts for UVC emitters may cause corona discharges, which may also cause ozone generation. Ozone may be generated by light having wavelengths below 242 nm. In some examples, ozone generation increases significantly with light having wavelengths below 200 nm. In some examples, one or more of the light sources disclosed herein may comprise filters to exclude extraneous wavelengths of light that may lead to ozone generation. For example, a light source having a peak wavelength of 222 nm may filter out other wavelengths of UV light above and/or below 222 nm. In some examples, the dose of 222 nm light may be kept below the exposure limits in order to limit ozone generation, with a higher dose of about 405 nm light used to have the same antimicrobial efficacy. In certain examples, the higher dose light used may have a wavelength of, for example, at least, greater than, less than, equal to, or any number in between about 400 nm, 401 nm, 402 nm, 403 nm, 404 nm, 405 nm, 406 nm, 407 nm, 408 nm, 409 nm and 410 nm. In some examples, one or more of the light sources disclosed herein may generate ozone below a 0.05 ppm (0.1 mg/m\hat{ }3) limit during heavy work that the ACGIH has set. In other examples, one or more of the light sources disclosed herein may generate ozone levels of, for example, at least, greater than, less than, equal to, or any number in between about 0.001 ppm, 0.002 ppm, 0.003 ppm, 0.004 ppm, 0.005 ppm, 0.006 ppm, 0.007 ppm, 0.008 ppm, 0.009 ppm, 0.01 ppm, 0.011 ppm, 0.012 ppm, 0.013 ppm, 0.014 ppm, 0.015 ppm, 0.016 ppm, 0.017 ppm, 0.018 ppm, 0.019 ppm, 0.02 ppm, 0.021 ppm, 0.022 ppm, 0.023 ppm, 0.024 ppm, 0.025 ppm, 0.026 ppm, 0.027 ppm, 0.028 ppm, 0.029 ppm, 0.03 ppm, 0.031 ppm, 0.032 ppm, 0.033 ppm, 0.034 ppm, 0.035 ppm, 0.036 ppm, 0.037 ppm, 0.038 ppm, 0.039 ppm, 0.04 ppm, 0.041 ppm, 0.042 ppm, 0.043 ppm, 0.044 ppm, 0.045 ppm, 0.046 ppm, 0.047 ppm, 0.048 ppm, 0.049 ppm, 0.05 ppm, 0.051 ppm, 0.052 ppm, 0.053 ppm, 0.054 ppm, 0.055 ppm, 0.056 ppm, 0.057 ppm, 0.058 ppm, 0.059 ppm, 0.06 ppm, 0.061 ppm, 0.062 ppm, 0.063 ppm, 0.064 ppm, 0.065 ppm, 0.066 ppm, 0.067 ppm, 0.068 ppm, 0.069 ppm, 0.07 ppm, 0.071 ppm, 0.072 ppm, 0.073 ppm, 0.074 ppm, 0.075 ppm, 0.076 ppm, 0.077 ppm, 0.078 ppm, 0.079 ppm, 0.08 ppm, 0.081 ppm, 0.082 ppm, 0.083 ppm, 0.084 ppm, 0.085 ppm, 0.086 ppm, 0.087 ppm, 0.088 ppm, 0.089 ppm, 0.09 ppm, 0.091 ppm, 0.092 ppm, 0.093 ppm, 0.094 ppm, 0.095 ppm, 0.096 ppm, 0.097 ppm, 0.098 ppm, 0.099 ppm and 0.1 ppm. In other examples, the irradiance and ozone generation related to the first light and/or second light may be modulated to limit exposure time or dosage to far-UVC emissions.

In some examples, one or more of the light sources disclosed herein may inactivate microorganisms/pathogens with light having a peak wavelength of light, or in some examples, multiple peak wavelengths, in a range of approximately 380 nm to approximately 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. In another example, one or more of the light source(s) disclosed herein may inactivate microorganisms/pathogens with light having a peak wavelength of light, or in some examples, multiple peak wavelengths, in a range of, at least, greater than, less than, equal to, or any number in between about 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm and 500 nm.

In some examples, one or more of the light sources disclosed herein may inactivate microorganisms/pathogens with light having a peak wavelength of light, or in some examples, multiple peak wavelengths, in a range of approximately 200 nm to approximately 230 nm, for example, approximately 222 nm light may be used as the peak wavelength. It should be understood that any wavelength within 200 nm to 230 nm may be utilized, and that the peak wavelength may include a specific wavelength plus or minus approximately 5 nm. In other examples the peak wavelength may be, for example, at least, greater than, less than, equal to, or any number in between about 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm and 230 nm.

In some examples, one or more of the light sources disclosed herein may inactivate microorganisms/pathogens with light having a peak wavelength of light, or in some examples, multiple peak wavelengths, in a range of approximately 100 nm to approximately 280 nm, for example, approximately 254 nm light may be used as the peak wavelength. It should be understood that any wavelength within 100 nm to 280 nm may be utilized, and that the peak wavelength may include a specific wavelength plus or minus approximately 5 nm. In other examples, the peak wavelength may be, for example, at least, greater than, less than, equal to, or any number in between about 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm and 280 nm.

In some examples, one or more of the light sources disclosed herein may use continuous disinfection. For example, an object or a surface intended to be disinfected may be continuously irradiated by one or more of the light sources disclosed herein. In some examples, an object or surface may be illuminated for a first percentage of time (e.g., 80% of the time) and not illuminated for a second percentage of time (e.g., 20% of the time), such as when the object or surface is being interacted with by a human, e.g., when using a toilet, when opening a garbage can, etc. In some examples, an integrated control system may determine that a minimum dosage over a certain period of time has been met for disinfecting purposes and disinfecting light may be turned off to save energy until the period of time expires and resets. In some examples, disinfecting light may be turned off 30% of the time over a specific time period, such as 24 hours, and may still be considered continuous (e.g., 16.8 hours out of 24). Other similar ratios may be possible.

In some examples, the objective may be to disinfect organisms or viruses in the air. Illumination of the air column or regions of the air in a room or space with the light sources disclosed herein may be used to disinfect the air. Similarly, the light sources disclosed herein may be used to disinfect water or other liquids that may be stationary or moving. The potentially improved efficacy as well as possible amelioration of concerns to impact on materials may be an advantage of the light sources that utilize more than one wavelength region as described herein.

In some examples, high use items such as cell phones may be difficult to continuously disinfect with external radiation. In some examples, one or more of the light sources disclosed herein may use intermittent disinfection. Some examples use intermittent disinfecting techniques where the disinfecting light may be only irradiating an object or surface intended to be disinfected, e.g., a cell phone, for certain period of time. In some examples, disinfecting light may shine on the object or surface intended to be disinfected for 8 hours overnight. In some examples, disinfecting light may shine on the object or surface intended to be disinfection for a period of time between 30 seconds and 8 hours. In some examples, the period of time the object or surface is exposed to the disinfecting light may match up with a specific time required to meet a certain dosage target for the inactivation of a specific microorganism.

In some examples, one or more of the light sources disclosed herein may pulse disinfecting light. By pulsing the UV source or otherwise reducing its duty cycle below 100%, the dose and exposure may be decreased, and the lifetime of the emission source may be increased. Pulsed light at high irradiances may be more effective than continuous light at lower irradiances. In some examples, pulsed light may have higher exposure limits compared to a continuous light source. In some examples, pulsed light may be considered to be intermittent because the light will be on and off periodically. In some examples, however, pulsed light may be used continuously and thus may also be considered continuous disinfection due to the length of time that light is pulsed (e.g., light may be pulsed for 24 hours straight). In some examples, the light may be pulsed, for example, at least, greater than, less than, equal to, or any number in between about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours and 48 hours. In another example, the light may include a pulse repetition frequency of, for example, at least, greater than, less than, equal to, or any number in between about 1 to 9999 Hz.

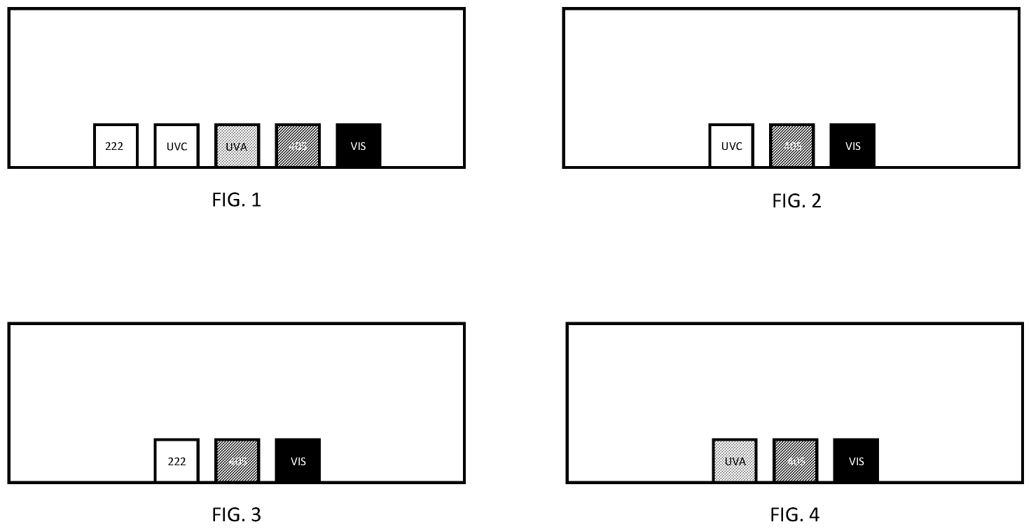

FIGS. 1-43 illustrate exemplary configurations of combined light disinfection devices in accordance with the present disclosure. In some examples, various light sources within the ranges of 100-280 nm, 200-230 nm, and/or 380-420 nm including, for example, UVA, UVC, visible, 222 nm, 254 nm, 260-270 nm, 280 nm, and/or 405 nm peak wavelength sources may be combined in one or more compartments with one or more filters, light converting materials, subcomponents, lens, beam collimators, controllers, and/or drivers to produce combined light disinfection devices that inactivate microorganisms including, for example, bacteria and viruses.

FIG. 1 illustrates an example device that may comprise one or more of the following: a 222 nm peak emission source, a UVC peak emission source, a UVA peak emission source, a 405 nm peak emission source, or a visible light peak emission source (VIS).

FIG. 2 illustrates an example device that may comprise a UVC peak emission source, a 405 nm peak emission source, and a visible light peak emission source (VIS).

FIG. 3 illustrates an example device that may comprise a 222 nm peak emission source, a 405 nm peak emission source, and a visible light peak emission source (VIS).

FIG. 4 illustrates an example device that may comprise a UVA peak emission source, a 405 nm peak emission source, and a visible light peak emission source (VIS).

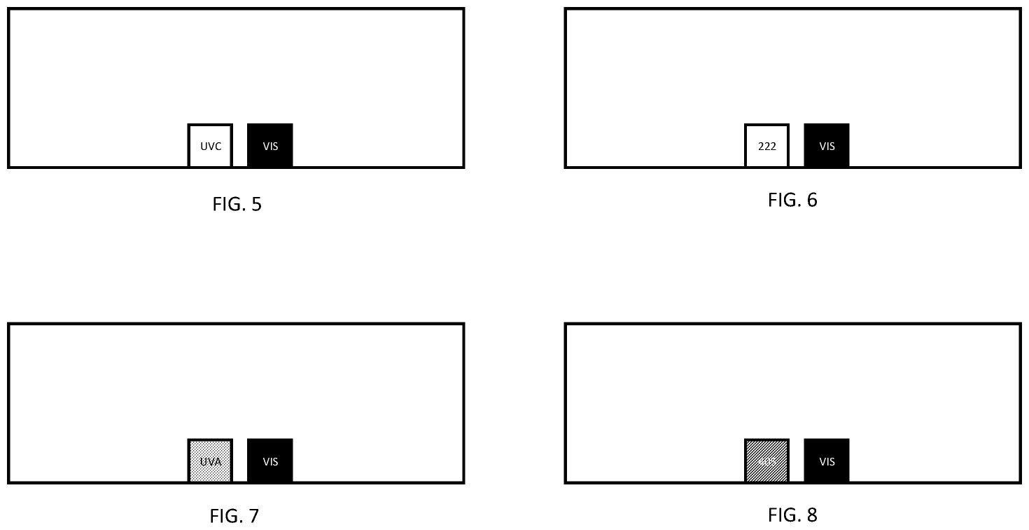

FIG. 5 illustrates an example device that may comprise a UVC peak emission source and a visible light peak emission source (VIS).

FIG. 6 illustrates an example device that may comprise a 222 nm peak emission source and a visible light peak emission source (VIS).

FIG. 7 illustrates an example device that may comprise a UVA peak emission source and a visible light peak emission source (VIS).

FIG. 8 illustrates an example device that may comprise a 405 nm peak emission source and a visible light peak emission source (VIS).

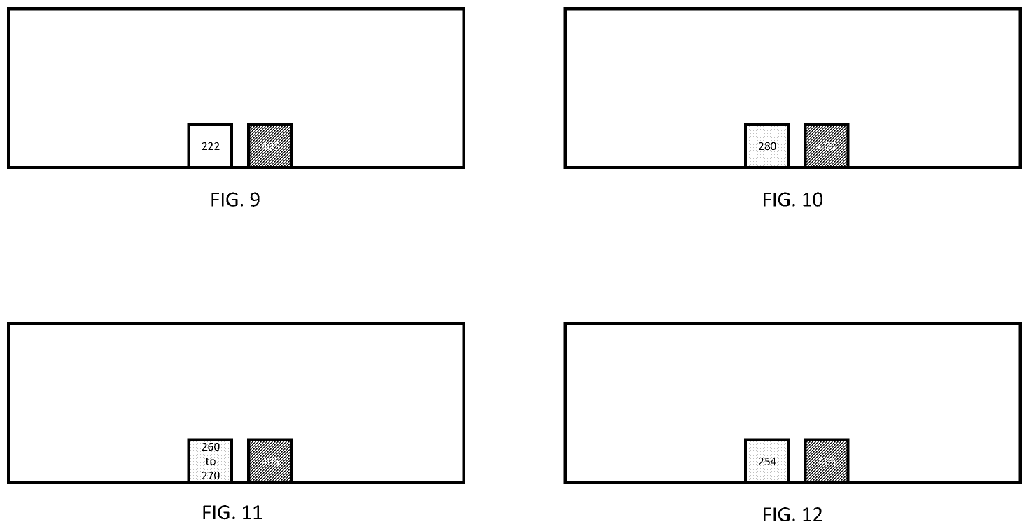

FIG. 9 illustrates an example device that may comprise a 222 nm peak emission source and a 405 nm peak emission source.

FIG. 10 illustrates an example device that may comprise a 280 nm peak emission source and a 405 nm peak emission source.

FIG. 11 illustrates an example device that may comprise an emission source with a peak range between 260 nm and 270 nm and a 405 nm peak emission source.

FIG. 12 illustrates an example device that may comprise a 254 nm peak emission source and a 405 nm peak emission source.

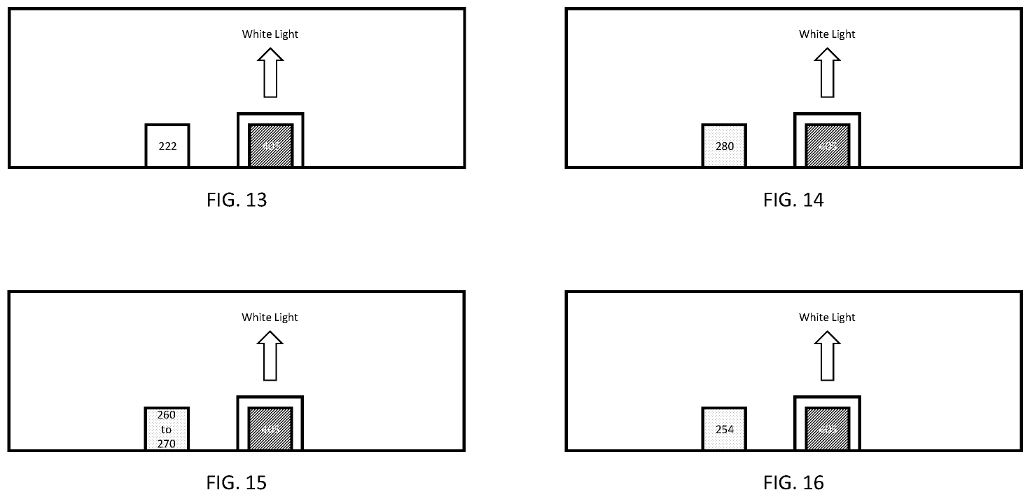

FIG. 13 illustrates an example device that may comprise a 222 nm peak emission source and a 405 nm peak emission source wherein the 405 nm peak emission source is embedded within a subcomponent configured to emit visible light wavelengths wherein the wavelengths combine to form white light.

FIG. 14 illustrates an example device that may comprise a 280 nm peak emission source and a 405 nm peak emission source wherein the 405 nm peak emission source is embedded within a subcomponent configured to emit visible light wavelengths wherein the wavelengths combine to form white light.

FIG. 15 illustrates an example device that may comprise an emission source with a peak between 260 nm and 270 nm and a 405 nm peak emission source wherein the 405 nm peak emission source is embedded within a subcomponent configured to emit visible light wavelengths wherein the wavelengths combine to form white light.

FIG. 16 illustrates an example device that may comprise a 254 nm peak emission source and a 405 nm peak emission source wherein the 405 nm peak emission source is embedded within a subcomponent configured to emit visible light wavelengths wherein the wavelengths combine to form white light.

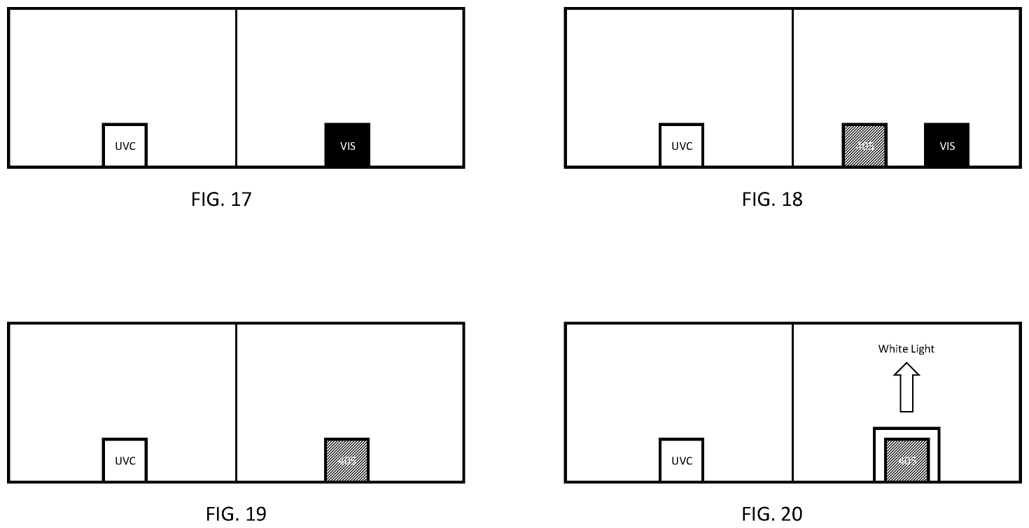

FIG. 17 illustrates the emission sources that may be separated within an example device such that the wavelengths from each emission source to not contact the other emission source. In some examples there that may be two compartments within an example device wherein one comprises a UVC peak emission source and the second comprises a visible light peak emission source (VIS).

FIG. 18 illustrates the emission sources that may be separated within an example device such that the wavelengths from each emission source to not contact the other emission source. In some examples there that may be two compartments within an example device wherein one comprises a UVC peak emission source and the second comprises a visible light peak emission source (VIS) and a 405 nm peak emission source.

FIG. 19 illustrates the emission sources that may be separated within an example device such that the wavelengths from each emission source to not contact the other emission source. In some examples there that may be two compartments within an example device wherein one comprises a UVC peak emission source and the second comprises a 405 nm peak emission source.

FIG. 20 illustrates the emission sources that may be separated within an example device such that the wavelengths from each emission source to not contact the other emission source. In some examples there that may be two compartments within an example device wherein one comprises a UVC peak emission source and the second comprises a 405 nm peak emission source wherein the 405 nm peak emission source that may be embedded within a subcomponent configured to emit visible light wavelengths wherein the wavelengths that may combine to form white light.

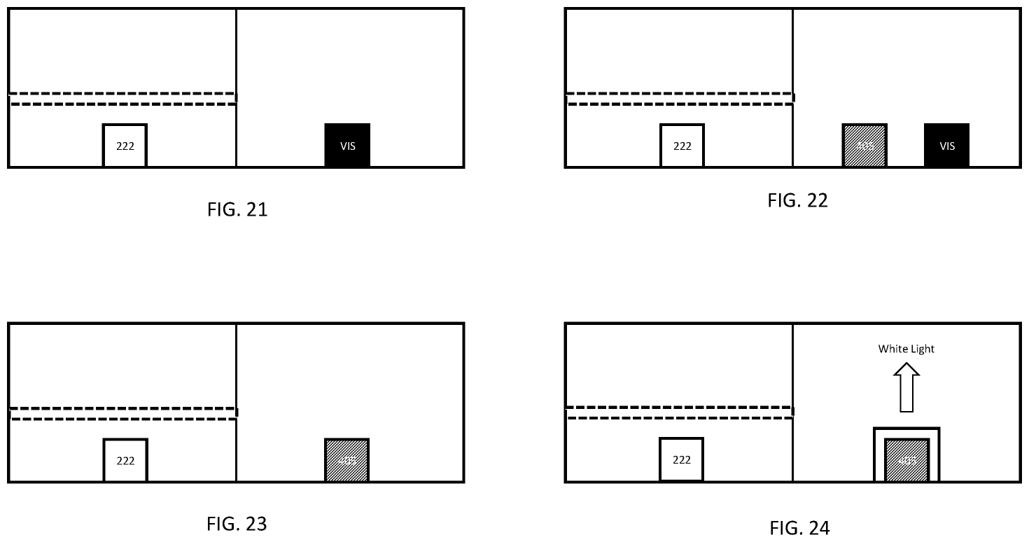

FIG. 21 illustrates the compartment comprising a 222 nm peak device that may additionally comprise a filter to filter out one or more wavelengths outside the range of 220 nm to 230 nm. In some examples the filter that may filter out one or more wavelengths outside the range of 217 nm to 227 nm. In some examples the filter that may filter out one or more wavelengths outside the range of 200 nm to 240 nm. In some examples the second compartment that may comprise a visible light peak emission source (VIS).

FIG. 22 illustrates the compartment comprising a 222 nm peak device that may additionally comprise a filter to filter out one or more wavelengths outside of a specified range including 222 nm. In some examples the second compartment that may comprise a visible light peak emission source (VIS) and a 405 nm peak emission source.

FIG. 23 illustrates the compartment comprising a 222 nm peak device that may additionally comprise a filter to filter out one or more wavelengths outside of a specified range including 222 nm. In some examples the second compartment that may comprise a 405 nm peak emission source.

FIG. 24 illustrates the compartment comprising a 222 nm peak device that may additionally comprise a filter to filter out one or more wavelengths outside of a specified range including 222 nm. In some examples the second compartment that may comprise a 405 nm peak emission source wherein the 405 nm peak emission source is embedded within a subcomponent configured to emit visible light wavelengths wherein the wavelengths combine to form white light.

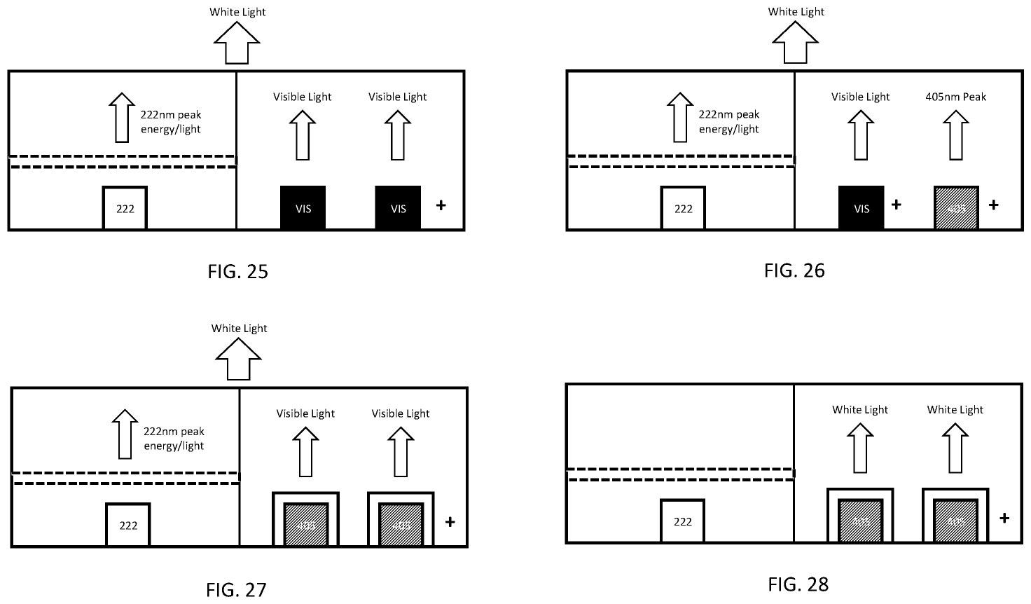

FIG. 25 illustrates the first compartment that may comprise a 222 nm peak emission source and the second compartment that may comprise one or more emission sources emitting visible light. When the emitted light/energy exits an example device it that may mix to form a combined white light.

FIG. 26 illustrates the first compartment that may comprise a 222 nm peak emission source and the second compartment that may comprise one or more emission sources emitting visible light and one or might emission sources emit 405 nm peak light. When the emitted light/energy exits an example device it that may mix to form a combined white light.

FIG. 27 illustrates the first compartment that may comprise a 222 nm peak emission source and the second compartment that may comprise one or more emission sources emitting visible light comprising a peak wavelength of 405 nm. When the emitted light/energy exits an example device it that may mix to form a combined white light.

FIG. 28 illustrates the first compartment that may comprise a 222 nm peak emission source and the second compartment that may comprise one or more emission sources emitting white light comprising a peak wavelength of 405 nm.

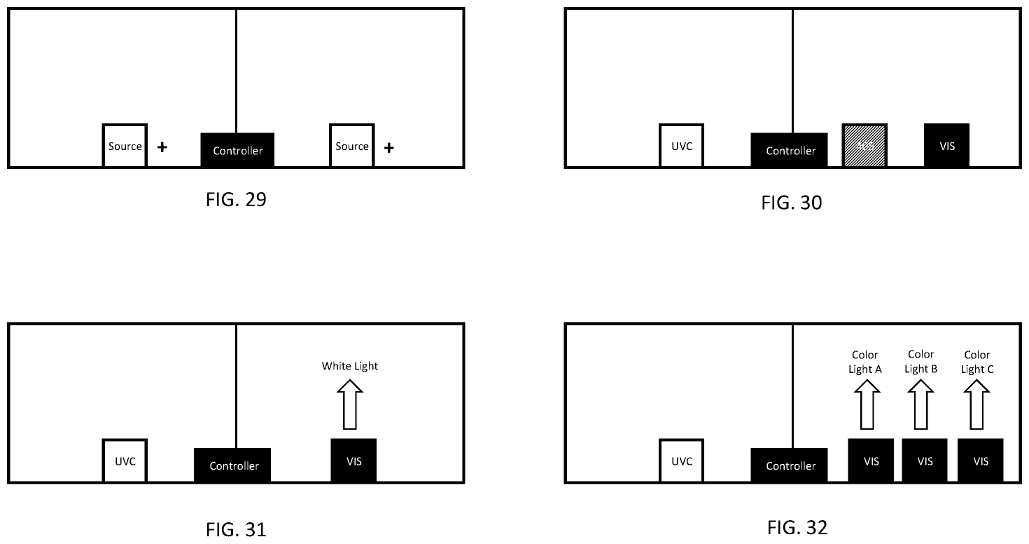

FIG. 29 illustrates an example device that may comprise two or more compartments each with one or more emission sources and a controller configured to independently control the emission sources within each compartment.

FIG. 30 illustrates an example device that may comprise two compartments. The first compartment that may comprise one or more UVC peak sources. The second compartment that may comprise one or more 405 nm peak sources and one or more visible light peak sources. an example device that may comprise a controller configured to independently control the emission sources within each compartment.

FIG. 31 illustrates an example device that may comprise two compartments. The first compartment that may comprise one or more UVC peak sources. The second compartment that may comprise one or more visible light peak emitters configured to produce white light. an example device that may comprise a controller configured to independently control the emission sources within each compartment.

FIG. 32 illustrates an example device that may comprise two compartments. The first compartment that may comprise one or more UVC peak sources. The second compartment that may comprise one or more visible light peak emitters configured to produce different peak wavelengths. an example device that may comprise a controller configured to independently control the emission sources within each compartment.

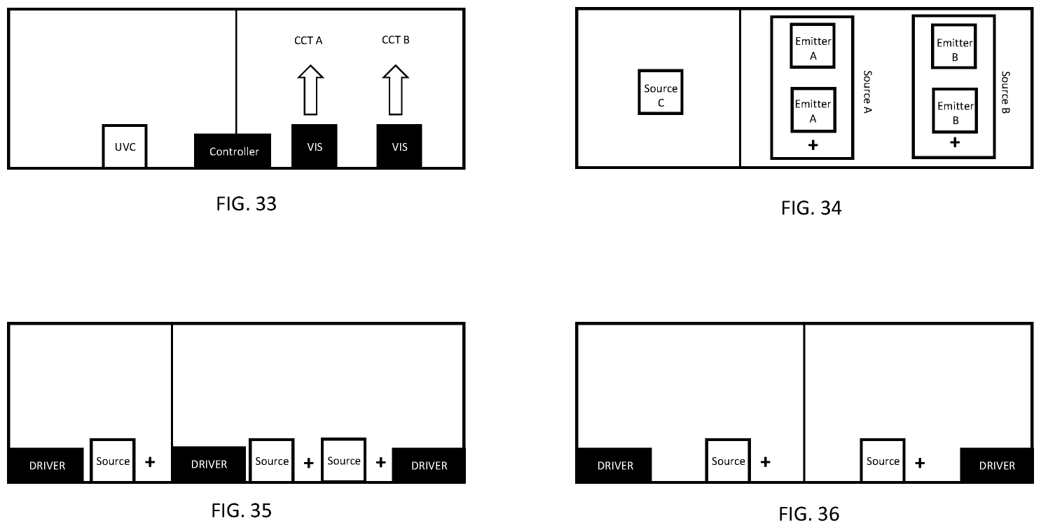

FIG. 33 illustrates an example device that may comprise two compartments. The first compartment that may comprise one or more UVC peak sources. The second compartment that may comprise one or more visible light peak emitters configured to produce white light with different CCT values. an example device that may comprise a controller configured to independently control the emission sources within each compartment.

FIG. 34 illustrates an emission source that may be made up of one or more emitters. In other examples, the emission source that may be made up of two or more emitters.

FIG. 35 illustrates an example device that may comprise two or more compartments each with one or more emission sources. The first compartment may comprise a driver configured to control the power provided to the sources within the first compartment. In other examples, the second compartment may comprise two or more different sources each with their own driver. In other examples, the emission source may be made up of multiple emitters.

FIG. 36 illustrates an example device that may comprise two or more compartments each with one or more emission sources and each with its own driver configured to control the power provided to the one or more sources within that compartment.

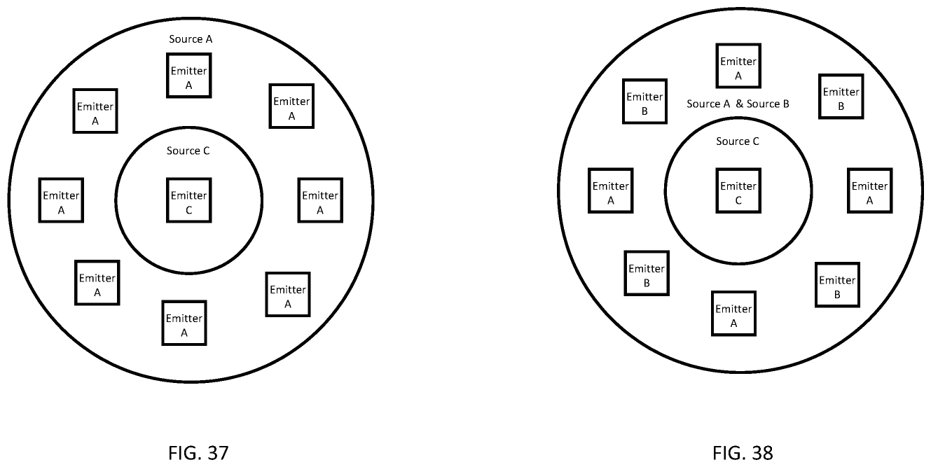

FIG. 37 illustrates a top view of an example device disclosed herein. The example device may comprise two compartments. In some examples, the example device may be round in shape, and may comprise a first center compartment and a second compartment surrounding the first center compartment. Each compartment may comprise a different emission source. The emission source that may comprise one or more emitters.

FIG. 38 illustrates a top view of an example device disclosed herein. In some examples, the example device may be round in shape, and may comprise a first center compartment and a second compartment surrounding the first center compartment. Each compartment may comprise a different emission source. The second compartment that may comprise two or more emission sources. The emission source that may comprise one or more emitters.

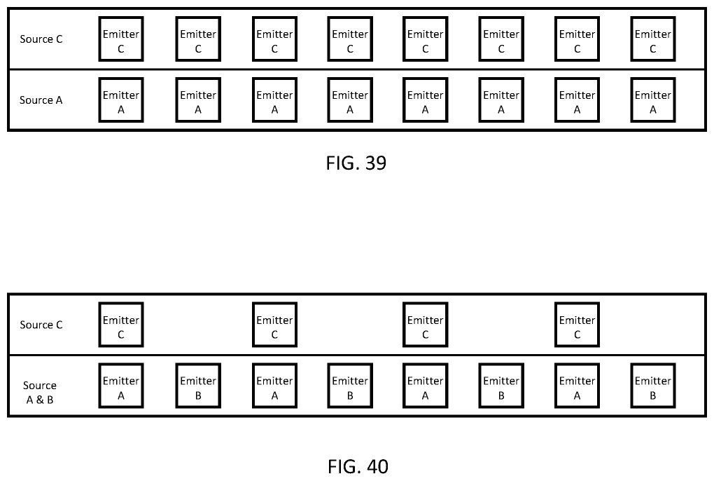

FIG. 39 illustrates a top view of another example device disclosed herein. The example device may include two compartments and the device may be a linear rectangle in shape. Each compartment that may comprise a different emission source. In some examples, the emission source that may comprise one or more emitters.

FIG. 40 illustrates a top view of another example device disclosed herein. The example device may include two compartments and the device may be a linear rectangle in shape. Each compartment that may comprise a different emission source. In some examples, the emission source may comprise one or more emitters. In another example, the emission source may comprise two or more emitters.

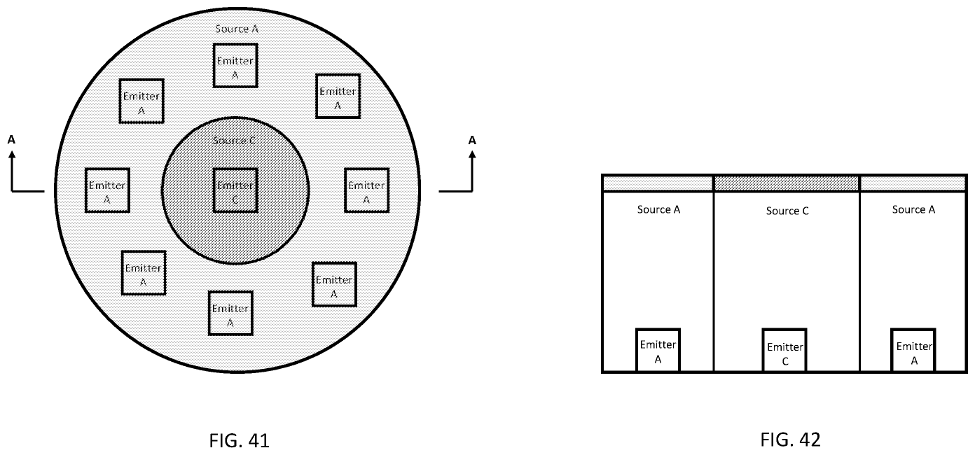

FIG. 41 illustrates a top view of another example device. The example device may include a lens material positioned over each compartment and may allow for the transmission of the emitter source(s) used within that compartment. The lens material for each compartment may be vary or may be similar in type.

FIG. 42 shows a cross-sectional view of an example device of FIG. 41 (section A-A).

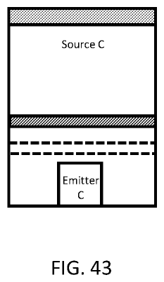

FIG. 43 illustrates a cross-sectional view of an example device comprising a remote filter and remote light converting material layer positioned above the emitters of Source C. The energy/light emitted from Emitter C may pass through the filter, then through a light converting material layer, and finally out the lens.

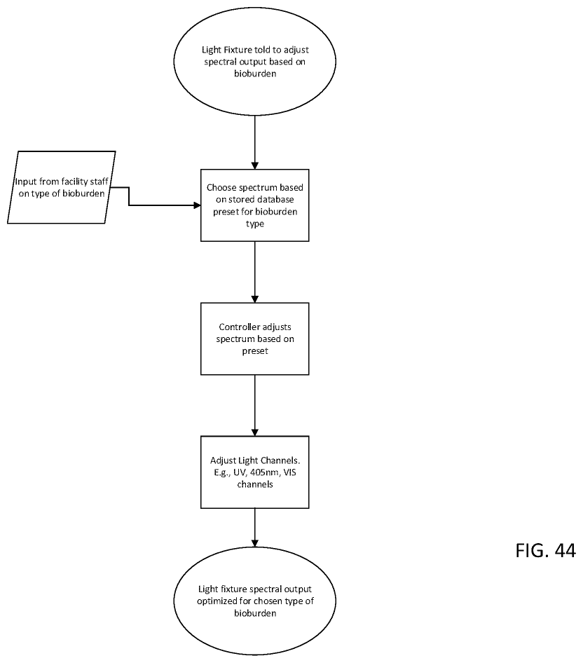

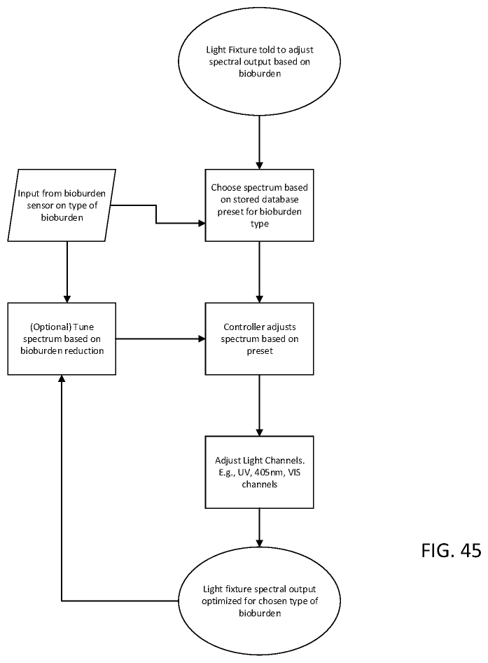

FIGS. 44-47 illustrate example flowcharts for various processes/algorithms that may be implemented by one or more controllers, drivers, or processors to control the exemplary configurations of combined light disinfection devices disclosed herein.

For example, FIG. 44 illustrates a process, that may be implemented by an integrated control system, to adjust the light's spectrum based on input from facility staff on the type of bioburden that is expected to be present, or by a sensor that determines the presence, amount, type, and/or concentration of microorganism or contaminant present.

FIG. 45 illustrates a process, that may be implemented by an integrated control system, to adjust the light's spectrum based on input from a bioburden sensor on the type, presence, amount, and/or concentration of bioburden that is detected. In some examples, the spectrum may be further tuned based on the efficacy of the spectrum as detected by the reduced presence of bioburden over time.

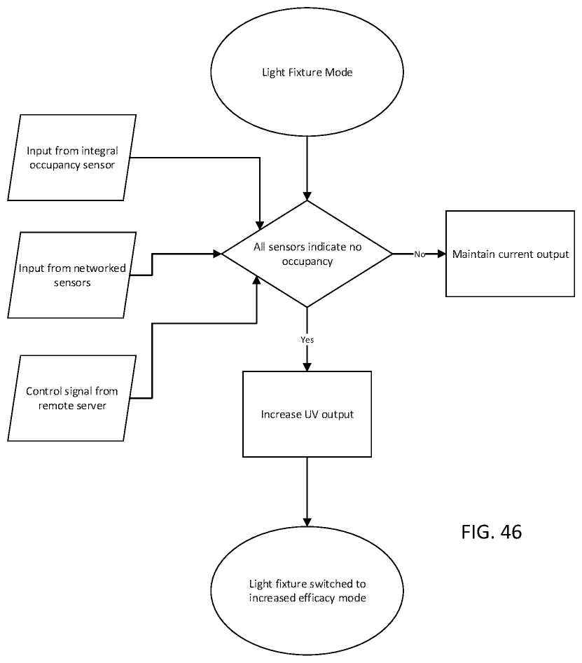

FIG. 46 illustrates a process, that may be implemented by an integrated control system, to adjust the light's UV output based on sensor and control signal input. All sensors may agree, for example, there is no occupancy before the light may switch to increased UV output.

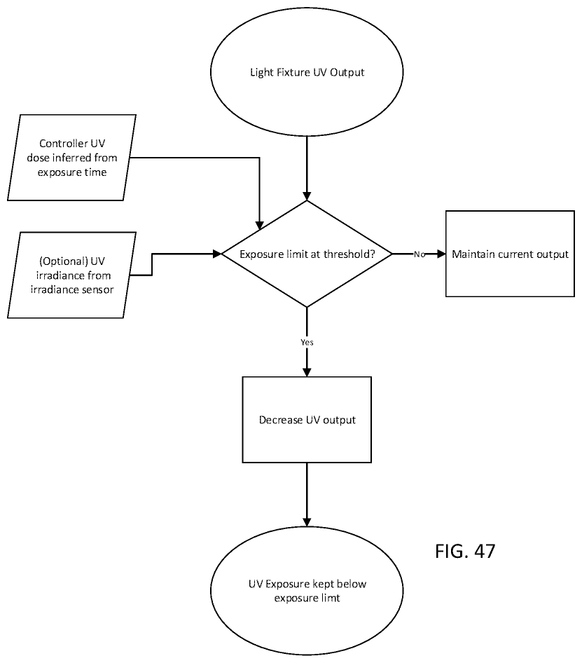

FIG. 47 illustrates a process, that may be implemented by an integrated control system, to adjust the light's UV output based on calculated or detected UV dosage. It may adjust the UV output such that UV exposure may not exceed some threshold. This threshold may be a defined exposure limit, or some other lower value.

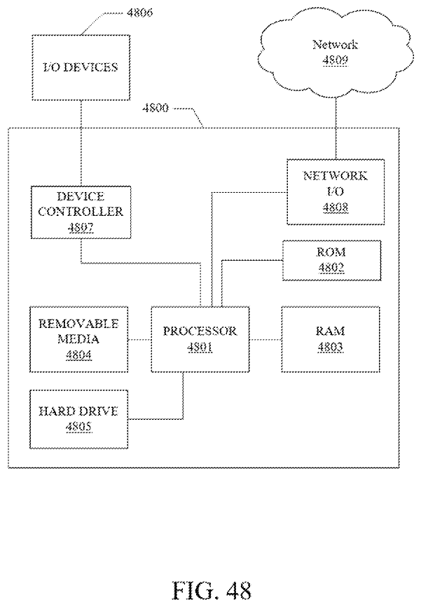

The controllers, drivers, processors and/or other computing devices described herein may be implemented via a hardware platform such as, for example, the computing device 4800 illustrated in FIG. 48. The components described above and elements described with reference to the computing device 4800 may be alternately implemented in software. The computing device 4800 may include one or more processors 4801, which may execute instructions of a computer program to perform any of the features described herein. The instructions may be stored in any type of tangible computer-readable medium or memory, to configure the operation of the processor 4801. As used herein, the term tangible computer-readable storage medium is expressly defined to include storage devices or storage discs and to exclude transmission media and propagating signals. For example, instructions may be stored in a read-only memory (ROM) 4802, random access memory (RAM) 4803, removable media 4804, such as a Universal Serial Bus (USB) drive, compact disk (CD) or digital versatile disk (DVD), floppy disk drive, or any other desired electronic storage medium. Instructions may also be stored in an attached (or internal) hard drive 4805. The computing device 4800 may include one or more input/output devices 4806, such as a display, touch screen, keyboard, mouse, microphone, software user interface, etc. The computing device 4800 may include one or more device controllers 4807 such as a video processor, keyboard controller, etc. The computing device 4800 may also include one or more network interfaces 4808, such as input/output circuits (such as a network card) to communicate with a network. The network interface 4808 may be a wired interface, wireless interface, or a combination thereof. One or more of the elements described above may be removed, rearranged, or supplemented without departing from the scope of the present disclosure. In some examples, the computing device 4800 may implement the processes/algorithms described with respect to FIGS. 44-47.

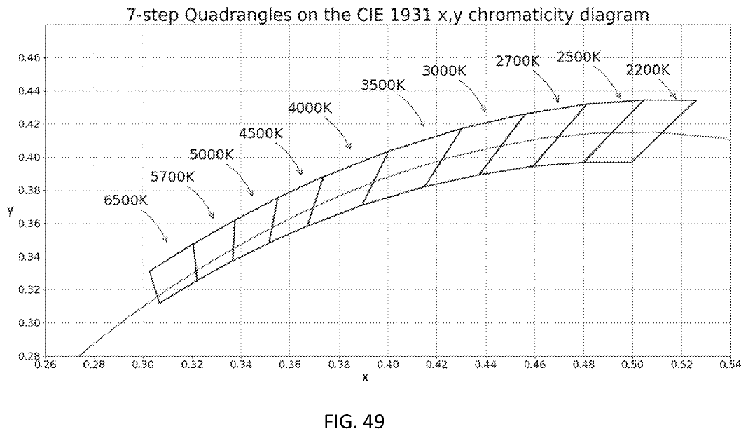

FIG. 49 illustrates ANSI C78.377-2017 white light standards with accepted x-y coordinates at selected CCTs, including 7-step MacAdam ellipses, and that shows quadrangles at various color temperatures for light-emitting devices in some examples of the disclosure. The ANSI C78.377-2017 standard states: “The purposes of this standard are, first, to specify the range of chromaticities recommended for general lighting with solid-state lighting products to ensure high-quality white light and, second, to categorize chromaticities with given tolerances so that the white light chromaticity of the products can be communicated to consumers.” Thus, the noted ANSI standard tries to define a chromaticity range (defined as “4-step” or “7-step” Quadrangles in the CIE 1931 x,y diagram, or the CIE 1976 u′,v′ diagram) for high quality white lights at different CCT values. The quadrangles set color consistency bounds so that LED to LED, or even fixture to fixture, lights look consistent. The 4-step or 7-step Quadrangles also help establish how far away from a particular CCT a light can be and still be considered that particular nominal CCT. In some examples, the device disclosed enables a disinfecting white light that can fall within the bounds of the Quadrangles at various color temperatures through the precise combination of selected emitters and light converting materials as described in examples of this disclosure. More specifically, the combined white light emitted from a light emitting device of the present disclosure can be quantified using (x,y) coordinates falling on the CIE 1931 Chromaticity diagram. The color temperature of the combined white light can vary between 1000 K to 8000 K for different examples. The (x,y) coordinates can be determined from a measured Spectral Power Distribution (SPD) graph of the emitted white light spectrum. When graphed, these determined (x,y) coordinates will fall within the bounds of a quadrangle for the color temperature of each example, and thus the combined light emitted can be defined as white light using the ANSI C78.377-2017 standard.

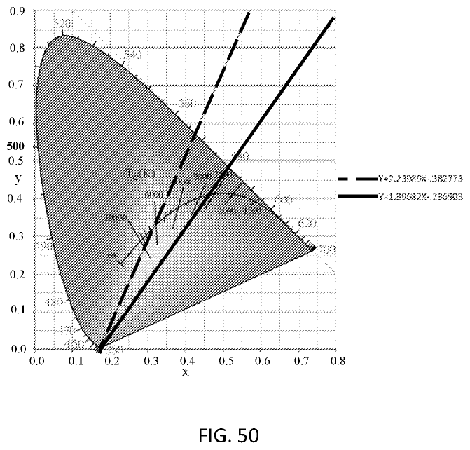

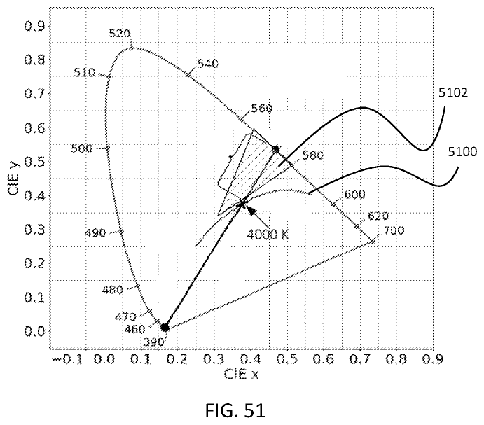

Additionally, white light according to some examples of the disclosure may be defined using the International Commission on Illumination (CIE) 1931 xy color space, which is a mathematically defined color space for which an equation defines the intended colors on a chromaticity diagram as illustrated in FIG. 50. Turning to FIG. 50, in these examples, white light may be defined as a combination of at least one first light source combined with at least one second light source in order to produce a combined light output that is perceived as a white light. The second light source(s) may comprise a color point, or an xy coordinate, on the CIE 1931 xy color space with a color point within the bounded area of the two lines shown, y=2.23989x−0.382773 and y=1.1551x−0.195082, and above the black body curve of the diagram. In some examples, the at least one second light source may comprise an xy coordinate on the International Commission on Illumination (CIE) 1931 xy color space diagram within a bounded area defined by a first line of approximately y=2.23989x−0.382773, a second line of approximately y=1.1551x−0.195082, and above a third line of approximately y=−2.57862x2+2.58744x−0.209201 (5100 in FIG. 51), which is within the above defined bounded area 5102 shown in FIG. 51. It should be understood that any one of the CRI value, CCT value, and CIE xy coordinate may be used alone or together in order to define white light according to examples of the disclosure.

In some examples, the device may include at least two emitters. The first emitter may emit a spectrum comprising a majority of the energy in the visible light spectrum, 380-700 nm. The second emitter may emit a spectrum comprising a majority of the energy in the non-visible ultraviolet spectrum, 100-380 nm. In some examples the first emitter may emit energy within the range of 380-420 nm. In some examples the first emitter may emit light within the range of 440-495 nm. In some examples the first emitter may emit light within the range of 420-440 nm. The first emitter may comprise one or more light converting materials configured to convert the emitted light to other wavelengths different from the light emitted from the light emitter. The exiting light from the combination of the first emitter and the one or more light converting materials may produce a combined light, the combined light being an off-white exiting light. In some examples, the second emitter may emit energy within the range of 100-700 nm. In some examples, one or more filters may be applied to the second emitter to block the majority of wavelengths outside of the range 200-230 nm. In some examples, not all energy may be blocked by the filter and some energy may pass into the visible light spectrum. In some examples, this passed through visible light may fall below the black body curve as defined by the CIE 1931 Chromaticity diagram. In some examples, the passed through visible light from the combination of the second emitter and the one or more filters combines with the off-white exiting light from the combined first emitter and one or more light converting materials to produce a high quality disinfecting white light.

The off-white light coordinates may be above the blackbody curve on the International Commission on Illumination (CIE) 1931 Chromaticity diagram (see FIG. 49 discussed above) in order to combine with the passed through light, whose coordinates are below the blackbody curve on the CIE 1931 Chromaticity diagram, to form a white light that falls on the blackbody curve or within the ANSI Quadrangles (e.g., defined by ANSI C78.377-2017). In some examples, the off-white exiting light coordinates may be above the boundary line:

y=−2.57862x²+2.58744x−0.209201

on the CIE 1931 Chromaticity diagram. In some examples, the passed through light range may be below the boundary line:

y=−2.57862x²+2.58744x−0.209201

on the CIE 1931 Chromaticity diagram. As noted, white light may correspond to the CIE 1931 Chromaticity diagram with coordinates within one of the quadrangles described by ANSI C78.377-2017, see FIG. 49.

In some examples, the combined light may have a proportion of spectral energy measured in an approximately 380 nm to approximately 420 nm wavelength range of greater than approximately 20%. In some examples, a combined light emitted by the light emitter(s) and the light-converting material(s) may be white and may have one or more of the following properties: (a) a proportion of spectral energy measured in an approximately 380 nm to approximately 420 nm wavelength range of greater than approximately 10%, (b) a correlated color temperature (CCT) value of 1000 K to 8000 K, (c) a color rendering index (CRI) value of 55 to 100, (d) a color fidelity (Rf) value of 60 to 100, or (e) a color gamut (Rg) value of 60 to 140.

The light may be configured with multiple emission sources that may be combined or used independently to provide inactivation/killing or microorganisms. For example, the light may contain one or more of the following: a 222 nm emission source, a UVC emission source, a UVA emission source, a 405 nm emission source, a visible light emission source. In some examples, the light device may contain a 222 nm emitter and a 405 nm LED. In some examples, the light device may contain a 280 nm LED and a 405 nm LED. The 405 nm LED may also emit other visible wavelengths and may be considered a white light source.

In some examples, emitters may emit wavelengths beyond their primary emission wavelength. A bandpass filter may be used to limit emission of wavelengths outside of the desired spectrum. For example, it may be desirable to use a bandpass or high-pass filter on a 222 nm broadband emission source to limit emission to only 222 nm+−10 nm (or some other number, e.g., 5 nm, 20 nm). In some examples, the light device may contain an ultra-low power 222 nm emission source and one or more 405 nm LEDs. The 222 nm emission source may output one to several mW of optical radiation, e.g., 1-100 mW. The example one or more of 405 nm LEDs may emit 1 mW to several Watts of optical radiation, e.g., 1-100,000 mW. The example one or more 405 nm LEDs may contain phosphors or other light converting materials to convert a portion of the 405 nm light to other wavelengths to generate white light. When combined with the bandpass filtered 222 nm light (which may or may not be visible), the overall light may be a white light. In some examples, the 405 nm LED and/or phosphors may be combined with unfiltered or partially filtered light from the 222 nm source to create white light. In some examples, visible light may be emitted from the 222 nm source and may contribute to the overall light to make it white. In some examples, the light may have a cutoff angle that avoids eye exposure. This may be achieved, for example, using lenses on the emission sources, lenses on the light fixture, or via a baffle or trim piece on the light fixture. The methods for controlling cutoff angle may only impact the 222 nm light, reducing incident UV exposure to the eyes of room occupants. The resulting cutoff angle, or beam angle, may be, for example, at least, greater than, less than, equal to, or any number in between about 5 degrees, 10 degrees 15 degrees, 20 degrees, 25 degrees, 30 degrees, 35 degrees, 40 degrees, 45 degrees, 50 degrees, 55 degrees, 60 degrees.

In some examples, the light device may have multiple independently controllable emission channels to enable spectral tunability. For example, the light device may contain one or more of the following channels: a UVC channel, a 405 nm channel, a visible light channel. In some examples, the light device may contain a 222 nm channel and a 405 nm channel. In some examples, the 405 nm channel may include other visible wavelengths. In some examples, the light device may contain a 222 nm channel and a white light LED channel. In some examples, the light device may contain a 222 nm channel and a plurality of visible light channels to allow for color or color temperature tuning. Such channels may be controlled by multiple independent drivers. Such drivers may in turn be controlled by a control system. The example control system may determine how much power to deliver to each emission source to effectively control the spectrum and output power per wavelength (spectral power) or wavelength range of the light.

In some examples, the control system may be operatively coupled to the light device. The example control system may be operative to control operational features of the device such as but not limited to: a duration of illumination, exiting light color, light intensity, and/or light irradiance. The control system may include any now known or later developed processor, microcontroller, system on a chip, computer, server, network device, mesh network device, internet-of-things device, etc. The light device may also include at least one sensor coupled to control system to provide feedback to control system. In some examples, sensor(s) may sense any parameter of the control environment of the device, including but not limited to: touch of the device, heat of a user's hand on the device, motion of a user, motion of structure to which device is coupled, temperature, light reception, and/or presence of microorganisms on exterior surface, etc. Sensor(s) may include any now known or later developed sensing devices for the desired parameter(s). The control system with sensor(s) (and without) can control operation to be continuous or intermittent based on external stimulus, and depending on the application.

In some examples, the control system and/or lights may be wired or wirelessly coupled to the internet (with or without a gateway) and a cloud or on-premises server to control or record data associated with the control system and/or lights. In some examples, usage patterns and determinations regarding time-on in different modes, irradiance or dosage thresholds being met may be recorded. In some examples, space utilization or social distancing records may be determined. This can be used for controlling them and recording use.

To limit exposure to safe levels when people or animals are present, a control system may be used to limit the dose provided by the light. The control system may calculate the allowable dose and/or irradiance based on the spectrum emitted by the light, e.g., there are different exposure limits for different wavelengths. If the control system detects the presence of people, using for example, an occupancy sensor (sound, IR, ultrasonic, camera, photodiode, etc.), it may decrease the irradiance of the light such that an exposure limit is not exceeded while people are present. For example, when people are present the light device may be configured to emit an irradiance that may not exceed the 8-hour exposure limit within an 8-hour period. When people are not detected, the control system may increase the irradiance to increase the killing or inactivation efficacy of the system. In some examples, the light device may be configured to emit light significantly below the exposure limit when people are present, e.g., 2, 10, or 100 times less than some exposure limit. When people are not detected, the control system may increase the irradiance of the light while still being below the 8-hour (or other time period) exposure limit. Thus, in some such examples, even if the control system fails to detect the presence of people or otherwise malfunctions, the light device will not expose people to an unsafe dose of the light. In another example, the spectrum of the light could be adjusted depending on the presence of people. For example, the control system may cause output of 405 nm and/or other visible wavelengths of light when people are present, to provide illumination and/or disinfection. In some examples, the control system may cause output of 405 nm light and 222 nm light for a first period of time when people are present. In some such examples, the control system may, based on a dose of 222 nm light satisfying a threshold, cause output of only 405 nm light for a second period of time when people are present, When people are not present, the control system may cause output of other wavelengths in the UV spectrum (e.g., 222 nm, 254 nm, 270 nm, or 280 nm) instead of or in addition to the 405 nm and/or other visible wavelengths to increase the killing/inactivation efficacy of the system.

The control system could include multiple levels of redundancy for detecting people or otherwise ensuring people are not exposed to unsafe levels of light. For example, each light may include its own occupancy sensor in addition to a networked occupancy sensor shared between lights. The control system may include logic to ensure that when a control signal from a remote source, e.g., a manual switch, an internet of things (IOT) control signal, etc. is received, it may not override the current decision from the light's own occupancy sensor. The control system may be configured such that all sensors must collectively be set to or agreed on detecting no occupancy/presence before a higher dose of light may be administered. In some examples, the control system may implement a voting system where all votes must indicate no occupancy before the control systems activates a higher dose of light. In some examples, the control system may have a default setting where the light may never exceed an exposure limit, or some other dose higher than an exposure limit, unless all connected sensors do not detect occupancy. Timers, buttons, and other controls may also be used to ensure that the light can only be commanded to increase dosage remotely by someone outside the space once occupancy is not detected.

The light device may be configured to be passively safe. For example, the light device may be configured (via the control system) such that the total dose a person may be exposed to is less than the 8-hour (or some other duration) exposure limit for the wavelengths emitted by the light. In some examples, the control system may calculate the distances to the light and/or run simulations to determine a maximum irradiance in a space. The control system may direct installation of the light based on the simulations such that the distance from the light to a surface or person satisfies a threshold distance. In some examples, if the distance satisfies the threshold, then the irradiance, and therefore the dose, may not exceed an exposure limit. In some examples, the light(s) may be installed such that no person/animal may be irradiated beyond an exposure limit under expected operating conditions. Simulations and/or distance calculations may be used to determine where to install the light to ensure safety. For example, UL 8802, Outline of Investigation for Germicidal Systems, sets the workplace height of a UV simulation to 7 feet above the ground to simulate the worst case scenario of a person standing directly under an overhead light.

In some examples, instead of an 8-hour timeframe, some other threshold may be used to ensure an additional safety factor, e.g., 2, 10 or 100 times than the 8-hour exposure timeframe. In some examples where occupancy is transient (e.g., no more than a threshold amount of time) the timeframe may be set lower. For example, when the light is installed in a bathroom, the timeframe could be set to 2 hours, such that a higher irradiance is applied.

In some examples, indications may be presented to alert users that an exposure limit is approaching. For example, the light may change colors or flash periodically. Additionally, or alternatively, the light may further comprise an indicator light to signal that UVC light is on.

Some microorganisms may respond differently to different wavelengths. In some examples, the control system may adjust the spectrum of the light based on the type of microorganism. For instance, some microorganisms may require high levels of 405 nm light, e.g., >1 mW/cm² for several hours, whereas the same microorganisms may only require 10 uW/cm² at 222 nm in a smaller time period (minutes) to achieve the same kill. Therefore, it may be beneficial to know the type of microorganism so that the spectrum can be tailored to it. In some examples, the control system may be pre-programed to target specific microorganisms. In some examples, data regarding dosage, irradiance, etc. for a specific microorganism may be input manually. In some examples, the control system or remote server may comprise a database containing optimal spectra for different types of microorganisms. In some examples, a bioburden sensor may be used to detect the type of microorganism and transmit information to the control system for targeting the microorganism. In some examples, the bioburden sensor may be an autofluorescence sensor, which may comprise a light emitter to cause excitation of the bioburden, and a sensor to measure the resulting emission from the bioburden. This bioburden sensor may interact with the control system or remote database to cause tuning of the light's spectrum.

In some examples, a light device may be integrated with a combination of light emitters. In some examples the light device may be a light fixture such as a downlight, troffer, bay luminaire, linear luminaire, high bay luminaire, surgical fixture, undercabinet lighting, task lighting, pendant mount fixture, etc. In some examples, the internal structure of the device may have one or more different compartments. Each compartment may have a different light emitter. In some examples, a first compartment may have a light emitter emitting light within the ultraviolet range 100-400 nm, and a second compartment may have a light emitter emitting light with a proportion of the spectral energy within the range of 380-420 nm. In some examples, the light comprising a proportion of spectral energy in the 380-420 nm range may be a white light. In some examples, there may be a third compartment with a third light emitter. In some examples, one of the compartments within the device may be located within a center of the device and the other compartment may create a ring around the center compartment (e.g., FIGS. 37-38, 41-42). The compartments may be separated to minimize the amount of light from one compartment that reaches the other compartment. In some examples the compartments may be fixed together within the device. In some examples the compartments may be separate from each other mechanically. In some examples, the compartments may be modular such that one compartment may be replaced without interfering with other compartments.

In some examples, UV light may be degrative to many materials including the materials that a UV emitter is made from or housed within. UV light may cause the emitter materials to discolor and become brittle, potentially leading to the emitter no longer functioning properly. In some examples, the light device may reduce internal reflections of UV light at a component level (e.g., within the emitter) and at a device level (e.g., within the compartment housing the emitter). Internal reflections and/or light blocked from exiting through the lens can reduce the efficacy of the device in addition to the degrative effects discussed above

In some examples, UV light may be blocked from reaching other compartments. In some examples, the geometry of a compartment containing a UV emitter may be designed so that a majority of the light leaves through the lens without reflecting back within the compartment. In some examples, the geometry of any compartment (including emitters in the visible light range as well) may be designed so that a majority of the light leaves through the lens without reflecting back within the compartment.

In some examples, the UV emitter may comprise a 90 degree beam angle. In some examples, the UV emitter may comprise a beam angle between 60 and 130 degrees. In other examples, the UV emitter may comprise a beam angle of for example, at least, greater than, less than, equal to, or any number in between about 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 and 150 degrees. In some examples the compartment containing the UV emitter may be cylindrical in shape.

In some examples, there may be a single UV emitter with a 90 degree beam angle located in the center of the bottom surface of the cylinder compartment. In some such examples, the emitter may have a depth of 2 in, the depth being measured from a bottom surface of the emitter to a point or surface of light emission. In some such examples, the diameter of the cylinder may be 4 inches. In some such examples, the depth of the cylinder may be at most 4 inches to ensure the full 90 degree beam angle of light exits the light device.

In some examples, there may be a single UV emitter with a 90 degree beam angle located in the center of a bottom surface of a cylinder compartment. In some such examples, the emitter may have a depth of 2 in. In some such examples, the diameter of the cylinder may be 6 inches. In some such examples, the depth of the cylinder may be at most 5 in to ensure the full 90 degree beam angle of light exits the light device.

In some examples, the diameter of the cylinder may be 4 inches. In some such examples, the depth of the emitter may be 0.08 inches and the beam angle of the single emitter placed in the center of the cylinder at the bottom surface may be 120 degrees. In some such examples, the depth of cylinder may be at most 1.235 inches to ensure the full 120 degree beam angle exists the light device.

In some examples, the diameter of the cylinder may be 4 inches. In some such examples there may multiple emitters, five for example, with a depth of 0.08 inches and a beam angle of 120 degrees spaced 0.8 inches from the center emitter. In some such examples, the depth of the cylinder may be at most 0.657 inches to ensure the full 120 degree beam angle exits the light device.

In some examples, additional light direction techniques such as integrated lenses, waveguides, beam collimators, etc. may be used to prevent internal reflections. In some examples, a monolithic lens may be used. In some examples, a reflector and monolithic lens may be used. Materials used for light direction may be able to transfer the wavelengths of light used by the emitter(s), within the UVC range, for example.

In some examples, a module capable of emitting ultraviolet light may be used as a subcomponent within a device. The module may comprise of one of more of the following: LED PCBA, emitter, emitter package, driver or ballast, control circuitry, safety sensors, lens, reflector, cover, or enclosure. An LED PCBA may be a printed circuit board with surface mount LEDs. The module may also include driving circuitry, for example, to regulate current and voltage going to the LEDs. An emitter may be a UV emission source that is not an LED. A safety sensor may be used to prevent accidental exposure to the UV light. The safety sensor may comprise of an occupancy sensor, a timer, a button, or a control signal from a remote sensor or control system. The module may be enclosed such that UV light does not leak out and is only emitted through the lens.

UV light blocking dividers may be used between UV light sources and discrete LED light emitters. UV light blocking dividers may prevent the photodegradation of adjacent LEDs or other materials due to high intensity UV radiation within the same fixture housing. The effect of photodegradation may be due to the close proximity of UV emitters to LED emitters and the high intensity of UV radiation at distances contained within the fixture housing. Risk of LED photodegradation due to UV radiation may be limited to inter-fixture distances and may not affect LEDs at the typical distance between multiple fixtures. UV light blocking dividers may also act as reflectors described herein.

Materials that allow transmission of UV light without photodegradation including silicone, fused-silica or fused-quartz glass may be used as protective or aesthetic covers between the UV light source and the environment external to the fixture. Materials that allow significant reflection of UV light without photodegradation including aluminum may be used as reflectors or dividers internal to the fixture housing described herein. Materials that allow absorption of UV light without photodegradation including silicone, polytetrafluoroethylene (PTFE), or plastics like Acrylonitrile butadiene styrene (ABS) may be used as UV light blocking dividers internal to the fixture housing described herein.

The UV emitter may use a driver to power the light device at the correct voltage, current, and frequency. The UV emitter driver may use the same common power source as the LED driver, and may utilize control circuitry to logically determine the state of, and/or power delivered to the UV emitter. This control circuitry may be shared between the UV emitter and the LED emitters. The UV emitter may utilize a driver topology that increases the source voltage to allow UV emitter operation. The UV emitter driver may include circuitry that enables digital or analog control of power delivered to the emitter. This control may allow the output of the UV emitter to be reduced from maximum output.

Bandpass and/or high-pass filters may be used over the emitter(s) to filter out undesired wavelengths. The filter(s) may, for example, comprise bandpass filters, high-pass filters and/or low-pass filters. The filter, for example, may be a bandpass filter with a 50 nm band, and may, for example, block/reduce wavelengths outside of a desired wavelength range. In some examples, a high-pass filter may be utilized instead of a bandpass filter. A high-pass filter may be used, for example, where it is known that the emitter has relatively little emission past the desired wavelengths. The filter, for example, may be a bandpass filter with a 10 nm band centered on 220 nm, such as Chroma ET220/10 bp.

Optical components may be used to direct UV radiation in a manner to reduce incident eye or skin exposure. A recessed mounting location of the UV light source may be used with UV reflecting or absorbing materials described herein to direct the UV light onto desired surfaces only. Collimating lenses may be used to direct the UV light in a uniform column of light thereby eliminating UV light emission at acute angles relative to the plane of the UV emitter. The light emitting devices disclosed herein may be modified by optics (e.g., a lens), reflectors, or other assembly components or materials (e.g., an encapsulant). In some examples, silicones may transmit UV wavelengths and may therefore be used as lenses both to direct light and to protect the emitter from the environment. Silicone may be formed into lenses and used as a dustproof or even waterproof cover or enclosure. Fused-silica or fused-quartz glass may also transmit UV wavelengths and may be used as a lens or cover. Fused-silica may have better resistance to aging and degradation caused by UV wavelengths compared to other materials. Lenses may be helpful to direct light to where it is needed, which can help reduce the overall power needed from the UV emission source(s). Polytetrafluoroethylene (PTFE), polished aluminum, and some plastics like Acrylonitrile butadiene styrene (ABS), may be suitable as reflector materials for UV.

In some examples, light-converting materials discussed herein may include phosphors, optical brighteners, a combination of phosphors, a combination of optical brighteners, or a combination of phosphor(s) and optical brightener(s). In some examples, the light-converting materials may be quantum dots, a phosphorescent material, a fluorophore, a fluorescent dye, a conductive polymer, or a combination of any one or more types of light-converting materials.

Phosphors and light converting materials used with ultraviolet light technologies may be used to convert UV wavelengths into visible light wavelengths. The visible light wavelengths emitted by the phosphors and light converting materials may include: a spectrum with peak in the range of 380 nm-420 nm to add to existing 380 nm-420 nm light emitted by LEDs in the same fixture housing, a spectrum summing to visible white light, or a spectrum of other visible light ranges. Phosphors and light converting materials used with ultraviolet light technologies may include Rare-Earth Triphosphors which mix red, green, and blue light producing phosphors, The phosphors or other light converting material may be deposited directly on the light emitter or may be remote or further removed from the light emitter. Light-converting materials can be deposited, for example, as conformal coatings, doped encapsulants or binder materials, and remote phosphors. A single phosphor or combination of phosphors may be used to achieve the wavelength ranges described in this paragraph.

Light-converting materials may include a broad category of materials, substances, or structures that may have the capability of absorbing a certain wavelength of light and re-emitting it as another wavelength of light. Light-converting materials may be different from light-emitting materials and light-transmitting/filtering materials. Light-emitting materials may include materials, substances, or structures/devices that convert a non ultraviolet-visible-infrared (UV-VIS-IR) form of energy into a UV-VIS-IR light emission. Non ultraviolet-visible-infrared (UV-VIS-IR) forms of energy may be, and are not limited to: electricity, chemical reactions/potentials, microwaves, electron beams, or radioactive decay. Light-converting materials may be contained in or deposited on a medium, making a light-converting medium. Light-converting materials, light-converting mediums, light-converting filters, phosphors, and other light converters may be used as disclosed herein.

In some examples, the light device may emit light according to a proportion of spectral energy. The proportion of spectral energy may be an amount of spectral energy within a specified wavelength range, i.e., 380-420 nm, divided by a total amount of spectral energy of the light. In some examples, the proportion of spectral energy may be a percentage.

Different colors of light may be emitted with a percentage (e.g., 20%) of their spectral power distribution within the wavelength range of 380-420 nm or within a UV wavelength range. In some examples, various colors of light may be emitted with a percentage of 30% to 100% spectral power distribution within the wavelength range of 380-420 nm. For example, a white light containing light across the visible light spectrum from 380-750 nm, may be used for disinfection purposes, with at least 20% of its energy within the wavelength range of 380-420 nm.

In some examples, light exiting a light emitter may be white, may have a color rendering index (CRI) value of at least 70, may have a correlated color temperature (CCT) between approximately 2,500K and 5,000K and/or may have a proportion of spectral energy measured in the 380 nm to 420 nm wavelength range between 10% and 44%. Other colors (e.g., blue, green, red, etc.) may also be used with a minimum percentage of spectral energy (e.g., 20%) within the range of 380-420 nm, which provides the disinfecting energy. In some examples, the white light may include a proportion of spectral energy measured in the 200 nm to 230 nm wavelength range between 0.01% and 2%.

Light emitters producing visible light may take any light emitter form capable of emitting light e.g., light emitting diode (LED), LEDs with light-converting layer(s), laser, electroluminescent wires, electroluminescent sheets, flexible LEDs, organic light emitting diode (OLED), or a semiconductor die.

Light emitters producing ultraviolet or visible light may comprise, for example, an LED, an array of LEDs, a laser, an array of lasers, a vertical cavity surface emitting laser (VCSEL), or an array of VCSELs. Other light emitters that may be used may include, for example, any emitter capable of emitting ultraviolet light including LEDs, fluorescent lamps without phosphor coatings, xenon arc lamps, mercury vapor, short-wave UV lamps made with fused quartz, black lights (fluorescent lamp coated with UVA emitting phosphor), amalgam lamps, natural or filtered sunlight, incandescent lamps with coatings that absorb visible light, gas-discharge (argon, deuterium, xenon, mercury-xenon, metal-halide, arc lamps, planar microcavity microplasma), halogen lamps with fused quartz, solid-state lamps, excimer lamps (such as Krypton Chlorine), etc. In some examples, an LED emitter may comprise at least one semiconductor die and/or at least one semiconductor die packaged in combination with light converting materials. In some examples, the light emitter may be fitted with optical components that may alter the path of the light. (e.g., focus the light into a beam).

The device disclosed herein may be powered through power outlets, electrical power supplies, batteries or rechargeable batteries mounted in proximity to the appliance, and/or wireless or inductive charging. Where rechargeable batteries are employed, they may be recharged, for example, using AC power or solar panels (not shown), where sufficient sunlight may be available. In some examples, AC power and an AC to DC converter, i.e. LED driver or power supply, may be utilized. In some examples, direct DC power may be utilized when available.

In various examples described herein, light at a specified wavelength or wavelength range may correspond to light which has a maximum emitted energy/power/energy spectral density/power spectral density approximately at the specified wavelength or within the specified wavelength range, with reasonable variations (e.g., ±5 nm, ±10 nm, etc.).

The above discussed examples are simply examples, and modifications may be made as desired for different implementations. For example, steps and/or components may be subdivided, combined, rearranged, removed, and/or augmented; performed on a single device or a plurality of devices; performed in parallel, in series; or any combination thereof. Additional features may be added.

We claim:

1. A light fixture, comprising:
a first light source configured to emit a first light comprising a wavelength in a range of 380-420 nanometers (nm); and
a second light source configured to emit a second light comprising a wavelength in a range of 200-230 nm, wherein the first light source and the second light source are separated to prevent degradation, by the second light, of the first light source, and
wherein the first light and the second light combine to form a disinfecting light configured to initiate inactivation of microorganisms.

2. The light fixture of claim 1, wherein the disinfecting light is configured to initiate inactivation of microorganisms on a surface, in air, and in a liquid.

3. The light fixture of claim 1, wherein the first light source comprises at least one LED and wherein the second light source second light source is not an LED.

4. The light fixture of claim 1, further comprising a lens or aperture coupled to the second light source configured to create a cutoff angle of the second light, and wherein the cutoff angle prevents eye exposure to the second light.

5. The light fixture of claim 1, further comprising a third light source configured to emit a third light comprising a wavelength from about 250 nm to 290 nm.

6. The light fixture of claim 1, wherein the first light and the second light combine to form a white light.

7. The light fixture of claim 1, wherein the second light source emits the second light for a dose of about 250 J/m².

8. The light fixture of claim 7, wherein the exposure time minimizes ozone generation.

9. The light fixture of claim 8, wherein the ozone generation is less than 0.05 parts per million.

10. A system, comprising:
a first light source configured to emit a first light comprising a wavelength in a range of 380-420 nanometers (nm);
a second light source configured to emit a second light comprising a wavelength in a range of 200-230 nm, wherein the first light and the second light combine to form a disinfecting light configured to initiate inactivation of microorganisms; and
an integrated control system configured to determine a minimum dosage of the disinfecting light, and configured to determine a maximum dosage of UV light exposure.

11. The system of claim 10, further comprising a third light source configured to emit a third light comprising a wavelength of about 254 nm, or a white light.

12. The system of claim 10, wherein the disinfecting light is configured to initiate the inactivation of microorganisms on a surface, in air, and in a liquid.

13. The system of claim 10, wherein the integrated control system control is further configured to independently control the first light source, and the second light source.

14. The system of claim 13, wherein the integrated control system is further configured to limit the first light emission and the second light emission based upon an emitted wavelength and intensity of the first light source and the second light source.

15. The system of claim 13, further comprising a filter coupled to the second light source, wherein the filter is configured to block wavelengths outside of the range 200-230 nm.

16. The system of claim 13, wherein the second light source is further configured to pulse the emitted light.

17. The system of claim 13, wherein the first light source comprises a plurality of LEDs and wherein the second light source is not an LED.

18. A method for inactivating microorganisms, comprising:
providing a first light source configured to emit a first light comprising a wavelength in a range of 380-420 nanometers (nm);
providing a second light source configured to emit a second light comprising a wavelength in a range of 200-230 nm;
separating the first light source from the second light source to prevent degradation by the second light of the first light source;
combining the first light and the second light to form a disinfecting light, wherein the disinfecting light is configured to inactivate microorganisms on a surface, in air, and in a liquid; and
initiating inactivation of microorganisms.

19. The method of claim 18, wherein the second light source emits the second light for an exposure time of about 8-hours or less, wherein the exposure time minimizes ozone generation, and wherein the ozone generation is less than 0.05 parts per million (ppm).

20. The method of claim 18, further comprising providing a third light source configured to emit a third light comprising a wavelength of about 254 nm.
